(19) United States
(12) Patent Application Publication
(10) Pub. No.: US 2015/0152394 A1
(43) Pub. Date: Jun. 4, 2015

(54) FLAVIN-CONJUGATED GLUCOSE DEHYDROGENASE AND POLYNUCLEOTIDE ENCODING THE SAME

(71) Applicant: IKEDA FOOD RESEARCH CO., LTD., Fukuyama-shi, Hiroshima (JP)

(72) Inventors: Michinari Honda, Hiroshima (JP); Ryo Takenaka, Hiroshima (JP); Takafumi Takumi, Hiroshima (JP)

(21) Appl. No.: 14/389,495

(22) PCT Filed: Mar. 29, 2013
(86) PCT No.: PCT/JP2013/059639

§ 371 Date: Sep. 30, 2014

(30) Foreign Application Priority Data
Mar. 30, 2012 (JP) 2012-079835

(51) Int. Cl. C12N 9/04; C12Q 1/32; G01N 27/327; C12Q 1/54

(52) U.S. Cl. CPC C12N 9/0006; C12Q 1/54; C12Q 1/32; G01N 27/327; C12Y 101/9901

(57) ABSTRACT

The purpose of the invention is to provide flavin-conjugated glucose dehydrogenase having little variation in activity in the typical biosensor measurement temperature range (10-40° C.) and a method for measuring glucose using same. The present invention relates to flavin-conjugated glucose dehydrogenase having the following properties (1)-(3) and the like: (1) action: shows glucose dehydrogenase activity in the presence of an electron acceptor; (2) substrate specificity: the activity value is 10% or less relative to maltose, D-galactose, D-fructose, sorbitol, lactose, and sucrose when the activity value relative to D-glucose is taken to be 100%; and (3) temperature characteristics: the range of the activity value at 10-40° C. is 20-150% when the activity value at 30° C. is taken to be 100%.

### TECHNICAL FIELD

The present invention relates to a soluble flavin-conjugated glucose dehydrogenase (GLD) or the like which catalyzes a reaction dehydrogenating (oxidization) a hydroxyl group at position 1 of glucose. More specifically, the present invention relates to a novel GLD polypeptide, a polynucleotide encoding the same, a method for manufacturing the GLD, a method for measuring glucose characterized by using the GLD, a reagent composition for measuring glucose, a biosensor for measuring glucose, and the like.

### BACKGROUND ART

Rapid and correct measurement of a blood glucose concentration is important for diagnosing diabetes. Although methods for measuring glucose include chemical methods and enzymatic methods, the enzymatic methods are more excellent in terms of specificity and safety. Among the enzymatic methods, electrochemical biosensors are advantageous in terms of reduction of a sample amount, reduction of a measurement time and downsizing of a device.

As enzymes usable for such biosensors, glucose oxidases which comprise oxygen as an electron acceptor are known. However, since the glucose oxidases have problems that measurement errors are caused by dissolved oxygen in the blood, some glucose dehydrogenases which do not employ oxygen as the electron acceptor have been developed. Among the glucose dehydrogenases, flavin-conjugated glucose dehydrogenases require no addition of coenzymes and are not affected by dissolved oxygen, and thus they are attracting attention as enzymes for glucose biosensors (Patent Documents 1 to 7). These flavin-conjugated dehydrogenases may include an enzyme having an excellent substrate specificity (Patent Document 5), an enzyme which has an activity value of 15% or more at 10° C., an activity value of 30% or more at 20° C., an activity value of 70% or more at 60° C. when an activity value at 50° C. is taken to be 100% (Patent Document 6), a modified enzyme of a flavin-dependent glucose dehydrogenase derived from Aspergillus oryzae in which an activity value at 25° C. relative to the activity value at 37° C. taken to be 100% is improved in a disrupted cell suspension of a recombinant Escherichia coli (Patent Document 7), and the like.

### PRIOR ART DOCUMENTS

### Patent Documents

### Patent Document 1: Japanese Patent Application Publication No. 2007-289148 A

### Patent Document 2: International Publication WO 2007/139013

### Patent Document 3: International Publication WO 2008/001903

### Patent Document 4: International Publication WO 2004/058958

### Patent Document 5: International Publication WO 2010/140431

### Patent Document 6: Japanese Patent Application Publication No 2010-057427 A

### Patent Document 7: International Publication WO 2011/034108

### SUMMARY OF INVENTION

### Problem to be Solved

However, the activities of these conventional glucose dehydrogenase have wide fluctuations in the activities depending on the temperature zones such that, for example, the reactivity was high at a high temperature region, but low at a low temperature region. Therefore, even if the biosensor itself had a temperature-correcting function, in measuring a blood glucose level of a diabetes patient, a resulting value may have fluctuated depending on ambient temperatures, and thus an enzyme having a smaller fluctuation in activity in a wide temperature zone has been required.

Consequently, the problem of the present invention is to provide a flavin-conjugated glucose dehydrogenase which has a small fluctuation in activity in a measurement temperature range (10-40° C.) for a general biosensor, a method for measuring glucose using the same, and the like.

### Means to Solve the Problem

Thus, in the present invention, various microorganism-derived glucose dehydrogenases were searched, and it was found that, among filamentous fungus-derived glucose dehydrogenases, there were flavin-conjugated glucose dehydrogenases in which the substrate specificities to glucose were high and the activities at 10-40° C. were 20-150% when an activity value at 30° C. was taken to be 100%, and that the glucose concentration could be correctly measured with good reproducibility in a wider temperature range by using them, resulting in completion of the present invention.

That is, the present invention relates to the following aspects [1]-[14].

[1] A flavin-conjugated glucose dehydrogenase having the following properties (1)-(3):

(1) action: exhibiting glucose dehydrogenase activity in the presence of an electron acceptor;

(2) substrate specificity: an activity value for maltose, D-galactose, D-fructose, sorbitol, lactose and sucrose is 10% or less when the activity value for D-glucose is taken to be 100%; and
(3) temperature characteristics: the range of the activity value at 10-40° C. is 20-150% when the activity value at 30° C. is taken to be 100%.
   [2] The glucose dehydrogenase according to [1], wherein a molecular weight of a polypeptide of an enzyme protein is 60-70 kDa.
   [3] The glucose dehydrogenase according to [1] or [2], wherein an optimum temperature is 30-40° C.
   [4] The glucose dehydrogenase according to any one of [1]-[3], which is derived from a filamentous fungus.
   [5] The glucose dehydrogenase according to any one of [1]-[4], which is derived from a filamentous fungus belonging to Dothideomycetes.
   [6] A method for manufacturing the glucose dehydrogenase according to any one of [1]-[5], characterized in that a glucose dehydrogenase-producing bacterium belonging to filamentous fungi is cultured, and the glucose dehydrogenase is collected from the culture.
   [7] A glucose dehydrogenase having glucose dehydrogenase activity and consisting of the following protein (a), (b) or (c):

(a) a protein which has an amino acid sequence shown in SEQ ID NO: 2, 4, 6, 8, 10, 12, 14, 16, 18
(b) a protein which has an amino acid sequence shown in positions 17-591 of SEQ ID NO 2, positions 16-589 of SEQ ID NO 4, positions 24-592 of SEQ ID NO 6, positions 17-591 of SEQ ID NO 8, positions 18-586 of SEQ ID NO 10, positions 18-586 of SEQ ID NO: 12, positions 18-586 of SEQ ID NO: 14, positions 18-586 of SEQ ID NO 16, positions 18-586 of SEQ ID NO 18, or positions 18-586 of SEQ ID NO 20;

(c) a protein which has an amino acid sequence having at least 90% similarity with the amino acid sequence of (a) or (b), and has the glucose dehydrogenase activity.

[8] A polynucleotide consisting of the following (e), (f), (g) or (h):

(e) a polynucleotide which has a base sequence shown in SEQ ID NO 1, 3, 5, 7, 9, 11, 13, 15, 17 or 19;

(f) a polynucleotide which has an base sequence shown in positions 49-1773 of SEQ ID NO: 1, positions 46-1767 of SEQ ID NO: 3, positions 70-1776 of SEQ ID NO: 5, positions 49-1773 of SEQ ID NO: 7, positions 52-1758 of SEQ ID NO: 9, positions 52-1758 of SEQ ID NO: 11, positions 52-1758 of SEQ ID NO: 13, positions 52-1758 of SEQ ID NO: 15, positions 52-1758 of SEQ ID NO: 17, or positions 52-1758 of SEQ ID NO 19;

(g) a polynucleotide which hybridizes with the polynucleotide of (e) or (f) under stringent conditions and encodes a protein having the glucose dehydrogenase activity;

(h) a polynucleotide which encodes the protein described in (a)-(c).

[9] A recombinant vector containing the polynucleotide according to [8].
[10] A transformant containing the polynucleotide according to [8].
[11] A method for manufacturing glucose dehydrogenase, characterized in that the cell according to [10] is cultured, and glucose dehydrogenase is collected from the culture.
[12] A method for measuring glucose using a sample, and the glucose dehydrogenase according to any one of [1]-[5] or [7], or the glucose dehydrogenase manufactured by the method according to [6] or [11].
[13] A glucose measuring reagent containing the glucose dehydrogenase according to any one of [1]-[5] or [7], or the glucose dehydrogenase manufactured by the method according to [6] or [11].
[14] A biosensor for measuring glucose using the glucose dehydrogenase according to any one of [1]-[5] or [7], or the glucose dehydrogenase manufactured by the method according to [6] or [11].

### Advantages of the Invention

The present invention can provide a flavin-conjugated glucose dehydrogenase which has a small fluctuation in activity in a measurement temperature range (10-40° C.) for a general biosensor. The blood glucose level could be correctly measured with good reproducibility in a wider temperature range by using the enzyme.

### BRIEF DESCRIPTION OF DRAWINGS

FIG. 1 shows results of SDS-polyacrylamide gel electrophoresis for the glucose dehydrogenases (A) to (F) of the present invention.

FIG. 2 shows ranges of the optimum temperatures for the glucose dehydrogenases (A) to (F) of the present invention.

FIG. 3 shows results of the glucose amounts measured by the glucose dehydrogenases (A) to (F) of the present invention.

FIG. 4 shows pH stability of the glucose dehydrogenase (F) of the present invention.

### DESCRIPTION OF EMBODIMENTS

The glucose dehydrogenase of the present invention is a soluble flavin-conjugated glucose dehydrogenase which exhibits activity while being conjugated with a flavin as a coenzyme. Enzymes classified into EC1. 1. 99. 10 are exemplified. Herein, the flavin may include flavin adenine dinucleotide (FAD) and flavin mononucleotide (FMN).

The glucose dehydrogenase of the present invention characteristically has the following properties (1)-(3).

(1) Action: exhibiting glucose dehydrogenase activity in the presence of an electron acceptor.

(2) Substrate specificity: an activity value for maltose, D-galactose, D-fructose, sorbitol, lactose and sucrose is 10% or less when the activity value for D-glucose is taken to be 100%.

(3) Temperature characteristics: the range of the activity value at 10-40° C. is 20-150% when the activity value at 30° C. is taken to be 100%.

First, the glucose dehydrogenase of the present invention (1) exhibits glucose dehydrogenase activity in the presence of an electron acceptor, i.e. catalyzes a reaction of oxidizing a hydroxyl group of glucose to produce a glucono-δ-lactone in the presence of the electron acceptor. When the flavin-conjugated glucose dehydrogenase acts on glucose, the coenzyme FAD is converted into an FADH₂, and if there is a ferricyanide (e.g. [Fe(CN)₆]³⁻) as an electron acceptor, the FADH₂ converts it into a ferrocyanide (in this case, [Fe(CN)₆]⁴⁻) and returns to the FAD. When the ferrocyanide is subjected to an electrical potential, it imparts electrons to an electrode and returns to the ferricyanide. Therefore electrochemical signal detection is enabled by converting such an electron transfer substance into an electron acceptor.

Since the glucose dehydrogenase of the present invention has a high substrate specificity to D-glucose, it is suitable for measurement of glucose. The glucose dehydrogenase of the present invention (2) has lower reactivity for maltose, D-galactose, D-fructose, sorbitol, lactose and sucrose relative to, the reactivity for the D-glucose, and the activity value is 10% or lower, preferably 8% or lower, more preferably 6% or lower, and even more preferably 5% or lower when the activity value for the D-glucose is taken to be 100%. Furthermore preferably, the activity value for the D-fructose, sorbitol, lactose and sucrose is 1% or lower, particularly preferably 0.5% or lower when the activity value for the D-glucose is taken to be 100%.

For the temperature characteristics of the glucose dehydrogenase of the present invention, (3) the activity value at 10-40° C. is 20-150% when the activity value at 30° C. is taken to be 100%, and a lower limit of the activity value at 10-40° C. is preferably 30%, more preferably 40%, even more preferably 50%. Furthermore, an upper limit of the activity value at 10-40° C. is preferably 140%, more preferably 130%, even more preferably 120%, particularly preferably 110%.

That is, the suitable range is: preferably 30-130%, more preferably 40-120%, further preferably 50-100% when the substrate concentration is 10 mM; and preferably 30-140%, more preferably 40-130%, further preferably 50-120% when the substrate concentration is 50 mM.

Alternatively, the activity value at 10° C. is preferably 20% or more when the activity value at 30° C. is taken to be 100%: more preferably 30% or more, further preferably 40% or more, particularly preferably 50% or more when the substrate concentration is 10 mM; and more preferably 30% or more, further preferably 40% or more, particularly preferably 50% or more when the substrate concentration is 50 mM.

Alternatively, the activity value at 20° C. is preferably 40% or more when the activity value at 30° C. is taken to be 100%: more preferably 50% or more, further preferably 60% or more, particularly preferably 70% or more when the substrate concentration is 10 mM; and more preferably 50% or more, further preferably 60% or more, particularly preferably 70% or more when the substrate concentration is 50 mM.

Preferably, the glucose dehydrogenase of the present invention further has the following properties (4) to (6). That is, (4) a molecular weight of a polypeptide of an enzyme protein is preferably 60-70 kDa, more preferably 65-70 kDa. The molecular weight of the polypeptide of the enzyme protein means a molecular weight of a protein moiety from which sugar chains were removed, measured by a SDS-polyacrylamide gel electrophoresis method. For the molecular weight of whole enzyme measured by the SDS-polyacrylamide gel electrophoresis method, the molecular weight is changed as the amount of the added sugar chains is changed depending on its culture condition, purification condition, etc., and in the case of a recombinant enzyme, the presence or absence of the sugar chain and the amount of the added sugar are changed and the molecular weight varies also depending on its host cell or the like.

The glucose dehydrogenase of the present invention (5) preferably has an optimum temperature of 30-40° C. More specifically, in measuring the enzyme at various temperatures by a method for measuring enzyme activity mentioned below, the relative activity value at 30-40° C. is more preferably 50% or higher, even more preferably 60% or higher, most preferably 80° C. or higher when an activity value at a temperature where the enzyme exhibits the maximum activity is taken to be 100%.

The glucose dehydrogenase of the present invention (6) preferably has Km of 1-80 mM, or more preferably has Km of 5-60 mM.

The specific examples of the glucose dehydrogenase of the present invention include 6 kinds specified in the following examples 2-7.

Although the origin of the glucose dehydrogenase of the present invention is not particularly limited, it is preferably a filamentous fungus, more preferably a filamentous fungus belonging to Dothideomycetes, more preferably a filamentous fungus belonging to Dothideomycetidae or Pleosporomycetidae, more preferably a filamentous fungus belonging to Dothideales, Capnodiales or Pleosporales, even more preferably a filamentous fungus belonging to Dothioraceae, Davidiellaceae or Venturiaceae, particularly preferably any of a filamentous fungus belonging to Aureobasidium, Kabatiella, Cladosporium or Fusicladium, most preferably a filamentous fungus selected from Aureobasidium pullulans, Kabatiella caulivora, Kabatiella zeae, Cladosporium sp., Cladosporium cladosporioides, Cladosporium funiclosum, Cladosporium oxysporum, Cladosporium delicatulum, Cladosporium gossypiicola, Cladosporium tenuissimum and Fusicladium carpophilum. It specifically includes the 10 kinds of strain described in examples 1-11 in the present Specification.

The glucose dehydrogenase of the present invention can be produced by, for example, culturing a glucose dehydrogenase-producing bacterium belonging to filamentous fungi, and collecting the glucose dehydrogenase from the culture.

For culturing microorganisms used in the present invention, conventional medium for culturing microorganisms can be used. Either a synthesized medium or a natural medium may be used, as long as the medium moderately contains carbon sources, nitrogen sources, minerals and other micronutrients required by the microorganisms of use. As the carbon sources, glucose, sucrose, dextrin, starch, glycerol, molasses, etc. can be used. As the nitrogen sources, inorganic salts such as ammonium chloride, ammonium nitrate, ammonium sulfate and ammonium phosphate, amino acids such as DL-alanine and L-glutamic acid, nitrogen-containing natural products such as peptone, meat extract, yeast extract, malt extract and corn steep liquor can be used. As the minerals, monosodium phosphate, disodium phosphate, monopotassium phosphate, dipotassium phosphate, magnesium sulfate, ferric chloride, etc. can be used.

The culture for obtaining the glucose dehydrogenase of the present invention should be generally carried out under an aerobic condition by a method such as shake culture and aeration agitation, preferably at 20° C. to 50° C., in a range of pH 4 to pH 8. The incubation period is preferably 2 days to 10 days. Owing to culturing by such a method, the glucose dehydrogenase can be produced and accumulated in a culture, particularly a broth. Alternatively, the glucose dehydrogenase can also be produced and accumulated in a cultured microorganism by the above-mentioned culture method. Next, as the method for obtaining the glucose dehydrogenase from a culture, a conventional protein purification method can be used. This method is e.g. a method that a microorganism is cultured and then the microorganism is removed by centrifugation or the like to obtain a culture supernatant, or a method that a microorganism is cultured, then the cultured microorganism is obtained by centrifuging the broth, the cultured microorganism is crushed by an appropriate manner, and a supernatant is obtained from the crushed liquid by centrifugation or the like. The glucose dehydrogenase contained in these supernatants can be purified by combining appropriate purifying manipulations such as ultrafiltration, salt precipitation, solvent precipitation, dialysis, ion-exchange chromatography, hydrophobic adsorption chromatography, gel filtration, affinity chromatography and electrophoresis.

In addition, a solid medium can also be used for the culture for obtaining the glucose dehydrogenase of the present invention. The culture method is not particularly restricted, and a stationary culture may be adopted, and a rotary culture and a fluidized bed culture in which a culture is consistently mixed may also be adopted, but the stationary culture is preferable as a culture apparatus with a small facility investment. Next, as the method for obtaining the glucose dehydrogenase from the culture, a conventional protein purification method can be used. That is, an extractant such as water is added to the culture, stirred, then medium solid contents such as bran are removed by a separation method such as centrifugation and filtration to obtain an extract. Meanwhile, the glucose dehydrogenase accumulated in the fungus can be collected by a method that the culture residue from which the extract was obtained is ground with an abrasive such as sea sand, to which water or the like is added to extract the glucose dehydrogenase released from the fungus, or other methods. Alternatively, the whole glucose dehydrogenase can be obtained by a method that the whole culture is ground with an abrasive such as sea sand, to which water or the like is added to extract both the glucose dehydrogenase released from the fungus and the glucose dehydrogenase secreted into the medium at the same time, or other methods. The glucose dehydrogenase contained in these supernatants can be purified by combining appropriate purifying manipulations such as ultrafiltration, salt precipitation, solvent precipitation, dialysis, ion-exchange chromatography, hydrophobic adsorption chromatography, gel filtration, affinity chromatography and electrophoresis.

The glucose dehydrogenase of the present invention may be a synthesized glucose dehydrogenase or a recombinant glucose dehydrogenase obtained by genetic engineering. Those skilled in the art can easily obtain such a recombinant glucose dehydrogenase on the basis of the disclosure of the present invention. For example, the glucose dehydrogenase can be obtained by a synthetic method on the basis of an amino acid sequence of the glucose dehydrogenase of the present invention and a base sequence of genes encoding it, and can also be industrially manufactured by a genetic engineering that the gene segment of the glucose dehydrogenase genes is inserted into a known expression vector such as a commercial expression vector, the obtained plasmid is used to transform a host such as Escherichia coli and filamentous fungus, and the transformant is cultured to obtain the desired glucose dehydrogenase.

The glucose dehydrogenase of the present invention is a glucose dehydrogenase consisting of the following protein (a), (b), (c) or (d):

(a) a protein which has an amino acid sequence shown in SEQ ID NO 2, 4, 6, 8, 10, 12, 14, 16, 18 or 20;

(b) a protein which has an amino acid sequence shown in positions 17-591 of SEQ ID NO: 2, positions 16-589 of SEQ ID NO: 4, positions 17-592 of SEQ ID NO: 6, positions 17-591 of SEQ ID NO 8, positions 12-586 of SEQ ID NO 10, positions 12-586 of SEQ ID NO 12, positions 12-586 of SEQ ID NO 14, positions 12-586 of SEQ ID NO 16, positions 12-586 of SEQ ID NO 18, positions 12-586 of SEQ ID NO 20, positions 24-591 of SEQ ID NO 2, positions 18-589 of SEQ ID NO 4, positions 24-592 of SEQ ID NO 6, positions 24-591 of SEQ ID NO 8, positions 18-586 of SEQ ID NO 10, positions 18-586 of SEQ ID NO 12, positions 18-586 of SEQ ID NO 14, positions 18-586 of SEQ ID NO 16, positions 18-586 of SEQ ID NO 18, or positions 18-586 of SEQ ID NO: 20;

(c) a protein which has an amino acid sequence having at least 90% similarity, preferably at least 95% similarity with the amino acid sequence of (a) or (b), and has the glucose dehydrogenase activity (the similarity is based upon the values of similarity calculated by the homology analysis between amino acid sequences with GENETYX (SOFTWARE DEVELOPMENT CO., LTD.));
(d) a protein which has an amino acid sequence having at least 60% identity, preferably at least 65%, 70%, 75%, 80%, 85%, 90% or 95% identity with the amino acid sequence of (a) or (b), and has the glucose dehydrogenase activity (the identity is based upon the values of identity calculated by the homology analysis between amino acid sequences with GENETYX (SOFTWARE DEVELOPMENT CO., LTD))

All the proteins of (a) to (d) are flavin-conjugated glucose dehydrogenases preferably having the above-mentioned properties (1) to (3), more preferably having the properties (4) to (6).

Among the respective amino acid sequences, signal sequences are amino acid sequences of positions 1-16 of SEQ ID NO: 2, positions 1-23 of SEQ ID NO: 6 and positions 1-11 of SEQ ID NO 12. From the signal sequences of SEQ ID NOs: 2, 6 or 12, amino acid sequences of positions 1-15 or 1-22 of SEQ ID NO 4, positions 1-16 or 1-23 of SEQ ID NO 8, or positions 1-11 of SEQ ID NOs: 10, 14, 16, 18 or 20 can be expected to be signal sequences.

It should be noted that the recombinant strain-derived mature protein may be a protein consisting of modified amino acid sequences in which several amino acids are replaced in, added to or deleted from an N-terminal of a wild strain-derived enzyme (mature protein) described herein. Namely, in relation to the recombinant strain-derived mature protein, 1-25, 20 or less, 10 or less, 9 or less, 8 or less, 7 or less, or 6 or less of amino acids may be added to or replaced in the N-terminal of the wild strain derived mature protein. Alternatively, even if 1-10, 9 or less, 8 or less, 7 or less, or 6 or less of amino acids are deleted, there can be the above-mentioned glucose dehydrogenase activity.

The polynucleotide of the present invention is a polynucleotide comprising the following (e), (f), (g), (h) or (i):

(e) a polynucleotide which has a base sequence shown in SEQ ID NO: 1, 3, 5, 7, 9, 11, 13, 15, 17 or 19;

(f) a polynucleotide which has an base sequence shown in positions 49-1773 of SEQ ID NO: 1, positions 46-1767 of SEQ ID NO: 3, positions 49-1776 of SEQ ID NO: 5, positions 49-1773 of SEQ ID NO: 7, positions 34-1758 of SEQ ID NO: 9, positions 34-1758 of SEQ ID NO: 11, positions 34-1758 of SEQ ID NO: 13, positions 34-1758 of SEQ ID NO: 15, positions 34-1758 of SEQ ID NO: 17, positions 34-1758 of SEQ ID NO: 19, positions 70-1773 of SEQ ID NO: 1, positions 67-1767 of SEQ ID NO: 3, positions 70-1776 of SEQ ID NO: 5, positions 70-1773 of SEQ ID NO: 7, positions 52-1758 of SEQ ID NO: 9, positions 52-1758 of SEQ ID NO: 11, positions 52-1758 of SEQ ID NO: 13, positions 52-1758 of SEQ ID NO: 15, positions 52-1758 of SEQ ID NO: 17, or positions 52-1758 of SEQ ID NO: 19;

(g) a polynucleotide which hybridizes with the polynucleotide of (e) or (f) under stringent conditions and encodes a protein having the glucose dehydrogenase activity;

(h) a polynucleotide which encodes the protein described in (a)-(c);

(i) a polynucleotide which has at least 60% identity, preferably at least 65%, 70%, 75%, 80%, 85%, 90% or 95% identity with the polynucleotide of (e) or (f), and encodes a protein having the glucose dehydrogenase activity (the identity is based upon the values of identity calculated by the homology analysis between base sequences with GENETYX (SOFTWARE DEVELOPMENT CO., LTD.))

All the polynucleotides (e) to (i) encode the proteins having the glucose dehydrogenase activity.

It should be noted that, in the respective polynucleotides, signal sequences are encoded by base sequences of positions 1-48 of SEQ ID NO: 1, positions 1-69 of SEQ ID NO: 5 and positions 1-33 of SEQ ID NO: 11. From the base sequences encoding the signal sequences of SEQ ID NO: 1, 5 or 11, base sequences shown in positions 1-45 or 1-66 of SEQ ID NO: 3, positions 1-48 or 1-69 of SEQ ID NO: 7, or positions 1-33 of SEQ ID NO: 9, 13, 15, 17 or 19 can be expected.

Also, a polynucleotide encoding a protein consisting of a modified amino acid sequence in which several amino acids are replaced in, added to or deleted from an N-terminal of a wild strain-derived enzyme (mature protein) as mentioned above can be used.

A percentage of identity between the amino acid sequence and the base sequence can be calculated using a released or marketed software comprising a comparison algorithm in reference to standard sequences (sequences of (a), (b), (e) or (f) in the present invention) as reference sequences. For example, BLAST, FASTA or GENETYX (SOFTWARE DEVELOPMENT CO., LTD.) or GeneDoc or the like can be used, and they can be used in default parameters.

In the present invention, as the specific condition of the “hybridizes under stringent conditions” in hybridization, a condition can be exemplified that e.g. 50% of formamide, 5×SSC (150 mM of sodium chloride, 15 mM of trisodium citrate, 10 mM of sodium phosphate, 1 mM of ethylenediaminetetraacetic acid, pH 7.2), 5×Denhardt's solution, 0.1% of SDS, 10% of dextran sulfate, and 100 μg/mL of modified salmon sperm DNA were incubated at 42° C., and then the filter is washed in 0.2×SSC at 42° C.

Although the signal sequence of the polynucleotide of the present invention is as described above, it can be presumed also by comparing with e.g. a signal sequence of glucose dehydrogenase sequences derived from Aspergillus terreus described in WO 2006/101239 (amino acid sequences shown in positions 1-19 of SEQ ID No 2 in its publication), or by using a signal sequence-predicting site (Signal P: http://www.cbs.dtu.dk/services/SignalP/). Even a protein consisting of amino acid sequences from which the presumed signal sequences were deleted may have the glucose dehydrogenase activity.

Note that, in the present invention, the “polynucleotides” specifically include synthetic DNAs encoding the flavin-conjugated glucose dehydrogenase, chromosomal DNAs, cDNAs synthesized from mRNA, or polynucleotides obtained by PCR amplification using them as templates. The “polypeptides” mean compounds having amino acids linked by peptide bonds, which are molecules synthesized by intracellular ribosomes or artificially, and furthermore include compounds prepared by glycosylating them, compounds artificially chemically-modified, etc.

Although the origin of the chromosomal DNA or RNA is not particularly limited, it is preferably a filamentous fungus, more preferably a filamentous fungus belonging to Dothideomycetes, more preferably a filamentous fungus belonging to Dothideomycetidae or Pleosporomycetidae, more preferably a filamentous fungus belonging to Dothideales, Capnodiales or Pleosporales, even more preferably a filamentous fungus belonging to Dothioraceae, Davidiellaceae or Venturiaceae, particularly preferably a filamentous fungus belonging to Aureobasidium, Kabatiella, Cladosporium or Fusicladium, most preferably Aureobasidium pullulans, Kabatiella caulivora, Kabatiella zeae, Cladosporium sp., Cladosporium cladosporioides, Cladosporium funiclosum, Cladosporium oxysporum, Cladosporium delicatulum, Cladosporium gossypiicola, Cladosporium tenuissimum or Fusicladium carpophilum. A DNA or cDNA library can be prepared by extracting the chromosomal DNA or RNA from the filamentous fungus. Subsequently, a plurality of oligonucleotide probes or degenerating primers are produced on the basis of a DNA encoding a known flavin-conjugated glucose dehydrogenase, a DNA encoding a flavin-conjugated glucose dehydrogenase derived from Aspergillus terreus e.g. described in WO 2006/101239, a DNA encoding a flavin-conjugated glucose dehydrogenase derived from Aspergillus oryzae described in Patent Document 3, and/or an alignment which was compared for identity with SEQ ID NOs: 1, 3, 5, 7, 9, 11, 13, 15, 17 and 19. Alternatively, a probe or a primer may be produced by cleaving a DNA with an appropriate restriction enzyme. The polynucleotides of the present application can be obtained from the library by a routine procedure such as hybridization, PCR and RT-PCR, using the above-mentioned probe or the primer.

Specifically, a forward primer and a reverse primer for decoding the internal sequences are produced from sites having higher identity in the alignments to carry out PCR using the library as a template. For a forward primer, the sites of positions 238-260 and positions 394-417 of SEQ ID NO: 1 can be exemplified in which two bases are preferably coincident with each other on the amplification side (downstream), and which can be preferably designed to have approximately 15-40 base-sequence length. For a reverse primer, the sites of positions 1600-1625 and positions 1735-1757 of SEQ ID NO: 1 can be exemplified in which two bases are preferably coincident with each other on the amplification side (upstream), and which can be preferably designed to have approximately 15-40 base-sequence length. When PCR is carried out using the primers designed from these sites, an annealing temperature is set to be low, preferably 40-50° C., more preferably 40-45° C. The second step of PCR may be carried out using a primer set inside the primer set used in the first step. A size of the PCR product is predicted from the position of the bases used for the primer, and an corresponding PCR product is decoded. If the decoded PCR product exhibits at least 50% identity, preferably at least 60%, more preferably at least 70%, more preferably at least 80%, more preferably at least 85%, even more preferably at least 90%, particularly preferably at least 95% identity with the corresponding position of SEQ ID NO 1, 3, 5, 7, 9, 11, 13, 15, 17 or 19, the internal sequence of the gene of the present application would have been obtained. Subsequently, the whole length of the gene of the present invention can be obtained from the decoded internal sequence by a known method. In other words, a primer is designed for clarifying region around the initiation codon and the termination codon of the gene of the present invention, and the primer is used to perform a 5′-RACE method and a 3′-RACE method using the library as a template. As a result, the region around the initiation codon and the termination codon of the gene encoding the glucose dehydrogenase of the present invention can be clarified. Subsequently, a primer which can amplify the whole length gene from the initiation codon to the termination codon encoding the glucose dehydrogenase of the present invention is designed, and the primer is used to obtain the polynucleotides of the present invention using the library as a template.

Homology search of e.g. BLAST (blastp or tblastn), etc. is conducted for a published sequence having unknown functions by means of the amino acid sequence of (a) or (b), and the polynucleotides of the present invention can be obtained from a gene sequence encoding an amino acid sequence of 550-650 amino acid-sequence length which was hit with the identity of at least 55%, preferably at least 60%, 65%, 70%, 75%, 80%, 85%, 90% or 95%. A primer whose whole length sequence can be obtained from the published sequence is designed, and amplified by PCR or RT-PCR using the DNA or RNA of the gene sequence-derived strain as templates to obtain the polynucleotides. Furthermore, a recombinant protein can be obtained by a routine procedure using the polynucleotides obtained by the amplification to confirm the glucose dehydrogenase activity. The DNA or RNA can be obtained also from the same or congeneric species of the strain as of the gene sequence-derived strain.

The polynucleotide of the present invention can be produced through modification by means of a known mutation introduction method, a mutation introduction PCR method, etc. Also, it can be obtained from a chromosomal DNA and its cDNA library by a probe hybridization method using an oligonucleotide produced on the basis of information of a nucleotide sequence. In the hybridization, the above-mentioned polynucleotide can be obtained by varying the stringent conditions. The stringent conditions are defined by a salt concentration, a concentration of organic solvents (formaldehyde, etc.), a temperature condition, etc. in the hybridization and washing steps, and various conditions known to those skilled in the art as disclosed in e.g. U.S. Pat. No. 6,100,037, Specification, etc. can be adopted.

The polynucleotide of the present invention can be in-vitro synthesized by known chemical synthesis techniques such as those described in some literatures (for example, Carruthers (1982) Cold Spring Harbor Symp. Quant. Biol. 47: 411-418; Adams (1983) J. Am. Chem. Soc. 105: 661; Belousov (1997) Nucleic Acid Res. 25: 3440-3444; Frenkel (1995) Free Radic. Biol. Med. 19:373-380; Blommers (1994) Biochemistry 33: 7886-7896; Narang (1979) Meth. Enzymol. 68: 90; Brown (1979) Meth. Enzymol. 68: 109; Beaucage (1981) Tetra. Lett. 22: 1859; and U.S. Pat. No. 4,458,066).

The recombinant vectors of the present invention are cloning vectors or expression vectors, and among them an appropriate vector should be used according to a kind of the polynucleotide as an insert, its purpose of use, etc. For example, when the flavin-conjugated dehydrogenase is produced using a cDNA or its ORF region as an insert, an expression vector for in vitro transcription, as well as an expression vector respectively suitable for a prokaryotic cell such as Escherichia coli and Bacillus subtilis, a yeast, a filamentous fungus such as mold, and a eukaryotic cell such as insect cell and mammal cell, can be used. According to a host, a polynucleotide which has the same amino acid sequence as of the host but in which the codon usage is optimized may be introduced. Furthermore, although the host can be properly selected according to necessities of sugar chains and other peptide modifications, preferably a host capable of adding a sugar chain is selected to produce an enzyme having a sugar chain.

As the transformant of the present invention, e.g. a prokaryotic cell such as Escherichia coli and Bacillus subtilis, a yeast, and a eukaryotic cell such as mold, insect cell and mammal cell, etc. can be used. Although the mold is not particularly limited, bacteria belonging to preferably Pezizomycotina, more preferably Dothideomycetes, Eurotiomycetes, Leotiomycetes or Sordariomycetes, even more preferably Eurotiales, particularly preferably Aspergillus are used. These transformants can be prepared by introducing a recombinant vector into a cell by a known method such as electric punch method, calcium phosphate method, liposome method and DEAE dextran method. Specific examples of the recombinant vector and the transformant include the recombinant vector shown in the following Examples and a transformed Escherichia coli and a transformed mold which were transformed by this vector.

When the flavin-conjugated glucose dehydrogenase of the present invention is produced by expressing a DNA in a microorganism such as Escherichia coli, an expression vector in which the polynucleotide is recombined into an expression vector having an origin replicable in a microorganism, a promoter, a ribosome binding site, a DNA cloning site, a terminator sequence, etc. is produced, a host cell is transformed by this expression vector, and then the resulting transformant is cultured to mass-produce the flavin-conjugated glucose dehydrogenase in the microorganism. At this time, an initiation codon and a termination codon are added ahead of and behind any translation area for expression, thereby a flavin-conjugated glucose dehydrogenase fragment including any area can also be obtained. Particularly, when a recombinant protein is expressed by a gram-negative bacterium such as Escherichia coli using a foreign gene containing a gene sequence encoding a secretory signal sequence, the recombinant protein is shifted to a periplasm, and therefore productivity is low. Thus, if the recombinant protein is intended to be efficiently collected, a sequence from which a gene sequence encoding the signal sequence was deleted should be used. Alternatively, the recombinant protein can be expressed as a fusion protein with other proteins. When the recombinant protein is produced by expressing it in a prokaryotic cell, the glucose dehydrogenase gene including no intron should be inserted, and particularly in the case of gram-negative bacteria, preferably a polynucleotide including no intron and no sequence encoding the signal sequence, e.g. a polynucleotide in which an initiation codon ATG is added to the polynucleotide described in (f) is inserted. In the case of gram-negative bacteria, it may be a polynucleotide including a sequence encoding the signal sequence, or a polynucleotide including no sequence encoding the signal sequence, e.g. a polynucleotide in which an initiation codon ATG is added to the polynucleotide described in (f), or a polynucleotide in which a sequence encoding the signal sequence is replaced by a sequence suitable for the host may be inserted. An expression level of the recombinant protein may be improved by replacing a termination codon by a termination codon optimal for the host. The expression vector for Escherichia coli includes a pUC system, pBluescriptII, a pET expression system, a pGEX expression system, a pCold expression system, etc.

Meanwhile, when the flavin-conjugated glucose dehydrogenase is produced by expression in a eukaryotic cell, the above-mentioned polynucleotide is inserted into a eukaryotic expression vector having a promoter, a splicing region, a poly (A) addition site, etc. to produce a recombinant vector, which is inserted into a eukaryotic cell, thereby the flavin-conjugated glucose dehydrogenase can be produced in a eukaryotic cell. It can be held in a cell in a plasmid-like state, or held to be incorporated into chromosomes for hold. The polynucleotide to be inserted may be a polynucleotide including a sequence encoding the signal sequence, a polynucleotide including no sequence encoding the signal sequence, e.g. a polynucleotide in which an initiation codon ATG is added to the polynucleotide described in (f), or a polynucleotide in which a sequence encoding the signal sequence is replaced by e.g. a signal sequence suitable for the host. An expression level of the recombinant protein may be improved by substituting a termination codon by a termination codon optimal for the host. The expression vector includes pKA1, pCDM8, pSVK3, pSVL, pBK-CMV, pBK-RSV, EBV vector, pRS, pYE82, etc. Furthermore, if pIND/V5-His, pFLAG-CMV-2, pEGFP-N1, pEGFPC1, etc. are used as expression vectors, the flavin-conjugated glucose dehydrogenase polypeptide can also be expressed as a fusion protein to which various tags such as His tag, FLAG tag and GFP are added.

As mentioned above, the flavin-conjugated glucose dehydrogenase of the present invention may be produced in vitro by preparing an RNA by in vitro transcription from a vector having the polynucleotide of the present invention (cDNA or its translation region), and conducting in vitro translation using this RNA as a template.

When the flavin-conjugated glucose dehydrogenase of the present invention is produced by in vitro expression, the polynucleotide is inserted into a vector having a promoter capable of binding to an RNA polymerase to produce a recombinant vector, and this vector is added to an in vitro translation system such as a rabbit reticulocyte lysate and wheat germ extract, including an RNA polymerase corresponding to the promoter, thereby the flavin-conjugated glucose dehydrogenase can be produced in vitro. The promoter capable of binding to an RNA polymerase includes T3, T7, SP6, etc. Vectors containing these promoters include pKA1, pCDM8, pT3/T718, pT7/319, pBluescriptII, etc.

In measuring the activity of the enzyme of the present invention, the enzyme is optionally diluted to a final concentration of preferably 0.15-0.6 units/mL for use. Note that a unit of enzyme activity of the enzyme means an enzyme activity for oxidizing 1 μmol of glucose in one minute. The enzyme activity of the glucose dehydrogenase (GLD) of the present invention can be measured by the following method.

### (Method for Measuring Enzyme Activity)

Each solution was mixed according to the following procedure, and an absorbance was measured to evaluate the GLD activity.

1.00 mL of 100 mM potassium phosphate buffer (pH 6.0), 1.00 mL of 1 M D-glucose solution, 0.61 mL of ultrapure water, 0.14 mL of 3 mM 2,6-dichlorophenolindophenol (hereinafter called DCIP), and 0.20 mL of 3 mM 1-methoxy-5-methylphenazinium methylsulfate (hereinafter, called 1-m-PMS) were mixed, kept at 37° C. for 10 minutes, and then 0.05 mL of enzyme sample was added, and the reaction was initiated. For 5 minutes from the initiation of the reaction, a decrement per one minute of the absorbance at 600 nm (ΔA600) associated with progression of the enzyme reaction was measured to calculate the GLD activity from a straight part according to Formula 1. In this measurement, for the GLD activity, an enzyme amount for reducing 1 μmol of DCIP at 37° C., pH 6.0 per one minute was defined as 1U.

$$enzyme activity ( U \text{/} mL ) = \frac{- ( Δ A 600 - Δ A 600 blank ) \times 3.0 \times df}{10.8 \times 1.0 \times 0.05} \qquad [ Formula 1 ]$$

In the formula, 3.0 represents a liquid volume (mL) of the reaction reagent+the enzyme solution, 10.8 represents a molar extinction coefficient (mM⁻¹cm⁻¹) of DCIP at pH 6.0, 1.0 represents an optical path length (cm) of a cell, 0.05 represents a liquid volume (mL) of the enzyme solution, ΔA600blank represents a decrement of the absorbance at 600 nm per one minute in the case that the reaction is initiated by adding a solution used for dilution of the enzyme instead of the enzyme solution, and df represents a dilution ratio.

As mentioned above, the glucose dehydrogenase of the present invention is not affected, by oxygen, has high specificity to glucose, maintains high activity even at room temperature, and thus is useful as an enzyme for measuring glucose levels, particularly blood glucose levels. Glucose in a test sample can be measured by a step of bringing the test sample containing glucose, e.g. blood, into contact with the glucose dehydrogenase of the present invention.

The glucose dehydrogenase of the present invention can be used for a glucose measuring reagent. The measuring reagent optionally contains a bovine serum albumin (BSA) or egg albumin, a sugar (e.g. trehalose) or a sugar alcohol not interactive with the enzyme, a carboxyl group-containing compound, an alkaline earth metal compound, an ammonium salt, a thermal stabilizer selected from a group consisting of sulfate, proteins, etc., or any other components such as a buffer known to those skilled in the art, to enhance thermal stability and storage stability of the enzyme and reagent components. Furthermore, a known substance which reduces the influence from impurities affecting the measurement in the test sample can be contained in the measuring reagent.

The glucose dehydrogenase of the present invention can be used for a biosensor.

The biosensor of the present invention may be any sensor in which the glucose dehydrogenase of the present invention is used as an enzyme for a reaction layer. For example, the biosensor is produced by forming an electrode system on an insulating substrate using a method such as screen printing and vapor deposition and further by providing a measuring reagent containing an oxidoreductase and an electron acceptor. When a sample solution containing a substrate is brought into contact with the measuring reagent of this biosensor, the measuring reagent is dissolved, and the enzyme is reacted with the substrate, and according to this, the electron acceptor is reduced. After completion of the enzyme reaction, the reduced electron acceptor is electrochemically oxidized, and at this time, this biosensor can measure the concentration of the substrate in the sample solution from the obtained oxidation current value. Additionally, a biosensor in a style of detecting coloring intensity, pH change or the like can also be constituted. Various substance can be measured by selecting an enzyme reacting with a substance to be measured as a substrate through these biosensor. For example, by selecting the glucose dehydrogenase of the present invention, for an enzyme, a glucose dehydrogenase which can measure a glucose level in a sample solution and is not affected by the enzyme, can be produced.

As the electron acceptor of the biosensor, substances having excellent electron-giving and receiving abilities can be used. The substances having excellent electron-giving and receiving abilities are chemical substances and proteinous electronic mediators generally called “electron carrier”, “mediator” or “oxidoreduction mediator”, and for example an electron carrier, an oxidoreduction mediator, etc. described in Japanese Unexamined Patent Application No 2002-526759 may be used as chemical substances falling under these substances.

Furthermore, the glucose dehydrogenase of the present invention can be used for a bio battery. The bio battery of the present invention is composed of an anode electrode for oxidation reaction and a cathode electrode for reduction reaction, and optionally includes an electrolyte layer which separates between the anode and the cathode as required. An enzyme electrode containing the electron mediators and the glucose oxidoreductase or the fusant thereof is used for the anode electrode, electrons generated by oxydation of the substrate are collected on the electrode, and protons are generated. Meanwhile, an enzyme to be generally used for the cathode electrode may be used on the cathode side, for example laccase, ascorbate oxidase or bilirubin oxidase is used, and the proton generated on the anode side is reacted with oxygen to generate water. As the electrode, electrodes to be generally used for the bio battery, such as carbon, gold and platinum can be used.

### EXAMPLES

Hereinafter, the present invention will be specifically explained by Examples. However, the present invention is not limited by the following Examples as long as the present invention is not beyond its gist. Quantification of the glucose dehydrogenase activity in the following Examples was carried out according to the above-mentioned method.

### Example 1

### Obtaining the Flavin-Conjugated Glucose Dehydrogenase of the Present Invention

### (1) Confirmation of the GLD Activity

GLD-producing bacteria are searched from about 3800 strains in total of those isolated from the natural world and those purchased from a depositary institution of microorganisms (National Institute of Technology and Evaluation: 2-5-8, Kazusa-kamatari, Kisarazu city, Chiba, JAPAN, 292-0818). As a result, The GLD activity has been confirmed in the culture supernatants of Aureobasidium pullulans S20, Aureobasidium pullulans NBRC4464, Kabatiella caulivora NBRC7314, Kabatiella zeae NBRC9664, Cladosporium sp. T799, Cladosporium sp.T806, Cladosporium cladosporioides NBRC4459, Cladosporium funiclosum NBRC6537, Cladosporium oxysporum NBRC32511, and Fusicladium carpophilum NBRC9645.

(2) Pulification of A. pullulans S20-Derived GLD

500 mL of a liquid medium consisting of 1% (w/v) of glucose (NACALAI TESQUE, INC.), 2% (w/v) of soy flour (Showa Sangyo Co., Ltd.), 0.5% (w/v) of potassium dihydrogenphosphate (NACALAI TESQUE, INC.), 0.05% (w/v) of magnesium sulfate heptahydrate (NACALAI TESQUE, INC.), 0.17 g/L of hydroquinone (NACALAI TESQUE, INC.), 0.0006% (w/v) of xylidine (Wako Pure Chemical Industries, Ltd.), 0.15 mM of EDTA (Wako Pure Chemical Industries, Ltd.), 2 ng/mL of biotin (Wako Pure Chemical Industries, Ltd.), 0.4 μg/mL of (+)-calcium pantothenate (Wako Pure Chemical Industries, Ltd.), 2 μg/mL of inositol (Wako Pure Chemical Industries, Ltd.), 0.4 μg/mL of nicotinic acid (Wako Pure Chemical Industries, Ltd.), 0.4 μg/mL of thiamine hydrochloride (Wako Pure Chemical Industries, Ltd.), 0.2 μg/mL of p-aminobenzoic acid (Wako Pure Chemical Industries, Ltd.), 0.2 μg/mL of vitamin B2 (NACALAI TESQUE, INC.), 10 ng/mL of folic acid (Wako Pure Chemical Industries, Ltd.), and water was introduced into a 2000 mL Sakaguchi flask, and autoclaved at 121° C. for 20 minutes. To this cooled liquid medium, an A. pullulans S20 strain was inoculated, and shake-cultured at 15° C. for 14 days. After the culture, the broth was filtered with a filter cloth, the collected filtrate was centrifuged to collect the supernatant, and furthermore filtrated with a membrane filter (10 μm, Advantech Co., Ltd.) to collect the culture supernatant, and concentrated with an ultrafiltration membrane of 8,000 cutoff molecular weight (Millipore Corp.) to obtain a crude enzyme liquid.

The crude enzyme liquid was adjusted to be a 60% saturated ammonium sulfate solution, left to stand at 4° C. overnight, and then centrifuged to collect a supernatant.

The supernatant was passed through TOYOPEARL Butyl-650C (TOSOH CORPORATION) column previously equilibrated by a 50 mM potassium phosphate buffer (pH 7.0) containing 60% saturated ammonium sulfate to adsorb the enzyme thereto. The column was washed with the same buffer, and then the enzyme was eluted by a gradient elution method from the buffer to 50 mM potassium phosphate buffer (pH 6.0) to collect an active fraction. The collected active fraction was concentrated by an ultrafiltration membrane, desalinated, equilibrated with 10 mM potassium phosphate buffer (pH 7.0), and passed through a DEAE-Sephacryl (GE Healthcare) column previously equilibrated by the same, buffer to adsorb the enzyme thereto. The column was washed with the same buffer, and then the enzyme was eluted by a gradient elution method to 10 mM potassium phosphate buffer (pH 7.0) containing 0.2 M sodium chloride to collect an active fraction. The collected active fraction was concentrated by an ultrafiltration membrane, then desalinated, equilibrated with 10 mM potassium phosphate buffer (pH 7.0), and passed through a monoQ5/5 (GE Healthcare) column previously equilibrated by the same buffer to adsorb the enzyme thereto. The column was washed with the same buffer, and then the enzyme was eluted by a gradient elution method to 10 mM potassium phosphate buffer (pH 7.0) containing 1 M sodium chloride to collect an active fraction. The collected active fraction was concentrated by an ultrafiltration membrane, then desalinated, equilibrated with 10 mM potassium phosphate buffer (pH 7.0), and passed through HiLoad26/60 Superdex 200 pg (GE Healthcare) previously equilibrated by the same buffer, then purified by a gel filtration using the same buffer to collect an active fraction. The collected active fraction was concentrated with an ultrafiltration membrane of 8,000 cutoff molecular weight, and then water substitution was performed to obtain a wild strain-derived ApsGLD sample. A specific activity of the purified enzyme was 378 U/mg.

### Example 2

### Expression of the GLD Derived from A. Aureobasidium pullulans S 20 (ApsGLD) by Eukaryotic Cell

### (1) Culture of Fungus Cells

A liquid medium consisting of 1% (w/v) of glucose (NACALAI TESQUE, INC.), 2% (w/v) of defatted soybean (Showa Sangyo Co., Ltd.), 0.5% (w/v) of corn steep liquor (San-ei Sucrochemical Co., Ltd.), 0.1% (w/v) of magnesium sulfate heptahydrate (NACALAI TESQUE, INC.), and water was adjusted to pH 6.0, 150 mL of it was introduced into a 500 mL Sakaguchi flask, and autoclaved at 121° C. for 20 minutes. To this cooled liquid medium, an Aureobasidium pullulans S20 strain was inoculated, shake-cultured at 15° C. for 90 hours, and then moist fungus cells were collected by means of bleached cloth.

### (2) Isolation of the Total RNA

After 200 mg of the moist fungus cells were frozen at −80° C., 100 μg of the total RNA was extracted using ISOGENII (NIPPON GENE CO., LTD.).

(3) Preparation of a cDNA Library

A cDNA library was prepared from the total RNA by a reverse transcription reaction using a reverse transcriptase and an oligo dT primer with an adabtor sequence. “SMARTer RACE cDNA Amplification kit” (TAKARA BIO INC.) was used as a reaction reagent and reaction conditions was adopted to a protocol described in an operating manual.

### (4) Cloning of ApsGLD Gene

Using the cDNA library obtained in (3) as a template, an ApsGLD gene was amplified by PCR. For the primer, common sequences were analyzed from a plurality of GLD sequences previously clarified by the inventors, and primer-F1, primer-F2, primer-R1 and primer-R2 were designed by using degenerated bases so that even GLD sequence having low identity can be amplified on the basis of the common sequences. In the first step, using the cDNA library obtained in Example 2 (3) as a template, PCR was carried out by means of the primer-F1 and the primer-R1. In the second step, using the PCR product in the first step as a template, PCR was carried out using the primer-F2 and the primer-R2. The sequences of the PCR product were analyzed, and a primer for clarifying the regions around the initiation codon and the termination codon of the gene from the decoded internal sequences was designed to perform the 5′-RACE method and the 3′-RACE method. Finally, PCR was performed using a primer pair of the following primer-ApsF and primer-ApsR to obtain a DNA fragment containing Aureobasidium pullulans S20 strain-derived ApsGLD gene of whole chain length of 1,776 bp shown in SEQ ID NO 1. The amino acid sequences encoded by the gene sequence are shown in SEQ ID NO: 2.

It should be noted that, in the amino acid sequences of SEQ ID NO 2, a signal sequence was predicted by Signal P4.1, and 16 amino acids of positions 1-16 in the amino acid sequences of SEQ ID NO 2 could be predicted to be the signal sequence.

primer-F1:
5′-CGGCACTCAGATYGAYTGGGCRTA-3′
primer-F2:
5′-AAGTTGGGHAACAACMTCACMTGG-3′
primer-R1:
5′-ATGCGCTCRGCAGCTCTCTCVGC-3′
primer-R2:
5′-ACGCCACCGAGHTCCTYSGACATCAT-3′
primer-ApsF:
5′-(TGACCAATTCCGCAGCTCGTCAAA)ATGTATCGTTTACTCTCTACA
TTTG-3′
(in parentheses: transcription-enhancing factor)
primer-ApsR:
5′-CGCTTCTAGAGCATGCCTACTGGTGGCTAGCCTCGATAAC-3′
(underlined: restriction enzyme site (SphI))
primer-GLD-F:
5′-CTCCAAGTTAGTCGAC(TGACCAATTCCGCAGCTCGTCAAA)-3′
(underlined: restriction enzyme site (SalI), in
parentheses: transcription-enhancing factor)

### (5) Preparation of Plasmid Vector Containing ApsGLD Gene

A plasmid vector was prepared using an amylase-based modified promoter derived from Aspergillus oryzae described in Known Document 1 (heterologous gene expression system of Aspergillus, Toshitaka Minetoki, Chemistry and Biology, 38, 12, 831-838, 2000). First, using the DNA fragment obtained in (4) as a template, PCR was performed by means of a primer pair of the primer-ApsR and primer-GLD-F to amplify the ApsGLD gene. Next, the amplified ApsGLD gene was bonded to the downstream of the promoter of the vector to prepare a plasmid vector on which the gene could be expressed. This plasmid vector for expression was introduced into an Escherichia coli JM109 strain to transform it, the resulting transformant was cultured, and a plasmid was extracted from the collected fungus cells using Illustra plasmid-prep MINI Flow Kit (GE healthcare). The sequence of the insert in the plasmid was analyzed, and then the ApsGLD gene (SEQ ID NO 1) could be confirmed.

### (6) Acquisition of Transformant

Using the plasmid extracted in (5), a recombinant mold (Aspergillus oryzae) which produces the ApsGLD was produced according to methods described in Known Document 2 (Biosci. Biotech. Biochem., 61 (8), 1367-1369, 1997) and Known Document 3 (genetic engineering technique for koji-mold for sake, Katsuya Gomi, journal of Brewing Society of Japan, 494-502, 2000). The resulting recombinant strain was refined in Czapek-Dox solid medium. As a host for use, Aspergillus oryzae NS4 strain was used. This strain was bred in a brewing laboratory in 1997 as described in Known Document 2, and has been utilized for analysis of transcription factor, breeding of high-producing strains for various enzymes. Currently, its strain furnished by National Research Institute of Brewing (3-7-1, Kagamiyama, Higashi-hiroshima-city, Hiroshima-Pref. 739-0046, Japan) is available.

### (7) Confirmation of Recombinant Mold-Derived ApsGLD

10 mL of a liquid medium consisting of 2% (w/v) of Pinedex (Matsutani Chemical Industry Co., Ltd.), 1% (w/v) of tripton (Becton, Dickinson and Company), 0.5% (w/v) of potassium dihydrogenphosphate (NACALAI TESQUE, INC.), 0.05% (w/v) of magnesium sulfate heptahydrate (NACALAI TESQUE, INC.) and water was introduced into a large test tube (22 mm×200 mm), and autoclaved at 121° C. for 20 minutes. To this cooled liquid medium, the transformant obtained in (6) was inoculated, and shake-cultured at 30° C. for 4 days. After the culture, the supernatant was collected by centrifugation, GLD activity was measured using a plate reader according to the above-mentioned method for measuring GLD activity, and then the GLD activity of the present invention could be confirmed. The broth was filtered with a filter cloth, the collected filtrate was centrifuged to collect the supernatant, and furthermore filtrated with a membrane filter (10 μm, Advantech Co., Ltd.) to collect the culture supernatant, and concentrated with an ultrafiltration membrane of 10,000 cutoff molecular weight (Sartorius AG). This sample was used as a recombinant ApsGLD sample.

### Example 3

### Expression of GLD Derived from B. Aureobasidium pullulans NBRC4464 Strain (ApnGLD) by Eukaryotic Cell

### (1) Cloning of ApnGLD Gene

The ApnGLD gene was amplified by PCR using the cDNA library of A. pullulans NBRC4464 prepared according to the method described in Example 2 (1) to (3) as a template.

According to the method described in Example 2 (4), PCR in the first step and the second step, the 5′-RACE method and the 3′-RACE method were performed. Finally, PCR was performed using a primer pair of the following primer-ApnF and primer-ApnR to obtain a DNA fragment containing A. pullulans NBRC4464 strain-derived ApnGLD gene of whole chain length of 1,770 bp shown in SEQ ID NO 3. The amino acid sequences encoded by the gene are shown in SEQ ID NO 4.

It should be noted that, in the amino acid sequences of SEQ ID NO 4, a signal sequence was predicted by Signal P4.1, and 15 amino acids of positions 1-15 in the amino acid sequences of SEQ ID NO: 4 could be predicted to be the signal sequence.

primer-ApnF:
5′-(TGACCAATTCCGCAGCTCGTCAAA)ATGTTGGGACTTGCTACCCTC
GCCC-3′
(in parentheses: transcription-enhancing factor)
primer-ApnR:
5′-CGCTTCTAGAGCATGCTTAGTGACTGGCCTTGATGATATC-3′
(underlined: restriction enzyme site (SphI))

### (2) Preparation of Plasmid Vector Containing ApnGLD Gene

Using the DNA fragment obtained in (1) as a template, PCR was performed by means of a primer pair of the primer-ApnR and primer-GLD-F to amplify the ApnGLD gene. According to the method described in Example 2 (5), the amplified ApnGLD gene was bonded to the downstream of the promoter to prepare a plasmid vector on which the gene could be expressed. Furthermore, according to the method described in Example 2 (5), the plasmid was extracted and the sequence of the insert in the plasmid was analyzed, and then the ApnGLD gene (SEQ ID NO 3) could be confirmed.

### (3) Acquisition of Transformant

Using the plasmid extracted in (2), a recombinant mold (Aspergillus oryzae) which produces the ApnGLD was produced according to the method described in Example 2 (6). The resulting recombinant strain was refined in Czapek-Dox solid medium.

### (4) Confirmation of Recombinant Mold-Derived ApnGLD

the activity of the ApnGLD was measured according to the method described in Example 2 (7), and then the GLD activity of the present invention could be confirmed. According to the method described in Example 2 (7), a sample obtained by concentrating the broth by ultrafiltration membrane was used as a recombinant ApnGLD sample.

### Example 4

### Expression of GLD Derived from C. Kabatiella caulivora (KcGLD) by Eukaryotic Cell

### (1) Cloning of KcGLD Gene

The KcGLD gene was amplified by PCR using the cDNA library of K. pullulans NBRC4464 strain prepared according to the method described in Example 2 (1) to (3) as a template.

According to the method described in Example 2 (4), PCR in the first step and the second step, the 5′-RACE method and the 3′-RACE method were performed. Finally, PCR was performed using a primer pair of the following primer-KcF and primer-KcR to obtain a DNA fragment containing K. caulivora NBRC7314 strain-derived KcGLD gene of whole chain length of 1,779 bp shown in SEQ ID NO 5. The amino acid sequences encoded by the gene are shown in SEQ ID NO 6.

It should be noted that, in the amino acid sequences of SEQ ID NO 6, a signal sequence was predicted by Signal P4.1, and 16 amino acids of positions 1-16 in the amino acid sequences of SEQ ID NO 6 could be predicted to be the signal sequence.

primer-KcF:
5′-(TGACCAATTCCGCAGCTCGTCAAA)ATGTTGGGACAAGTTGCTGCT
CTCG-3′
(in parentheses: transcription-enhancing factor)
primer-KcR:
5′-CGCTTCTAGAGCATGCTTACAAGTGCTTGGCCTTGATGAG-3′
(underlined: restriction enzyme site (SphI))

### (2) Preparation of Plasmid Vector Containing KcGLD Gene

Using the DNA fragment obtained in (1) as a template, PCR was performed by means of a primer pair of the primer-KcR and primer-GLD-F to amplify the KcGLD gene. According to the method described in Example 2 (5), the amplified KcGLD gene was bonded to the downstream of the promoter to prepare a plasmid vector on which the gene could be expressed. Furthermore, according to the method described in Example 2 (5), the plasmid was extracted and the sequence of the insert in the plasmid was analyzed, and then the KcGLD gene (SEQ ID NO: 5) could be confirmed.

### (3) Acquisition of Transformant

Using the plasmid extracted in (2), a recombinant mold (Aspergillus oryzae) which produces the KcGLD was produced according to the method described in Example 2 (6). The resulting recombinant strain was refined in Czapek-Dox solid medium.

### (4) Confirmation of Recombinant Mold-Derived KcGLD

The activity of the KcGLD was measured according to the method described in Example 2 (7), and then the GLD activity of the present invention could be confirmed.

### (5) Pulification of KcGLD

150 mL of a liquid medium consisting of 2% (w/v) of Pinedex (Matsutani Chemical Industry Co, Ltd.), 1% (w/v) of tripton (Becton, Dickinson and Company), 0.5% (w/v) of potassium dihydrogenphosphate (NACALAI TESQUE, INC.), 0.05% (w/v) of magnesium sulfate heptahydrate (NACALAI TESQUE, INC.) and water was introduced into a 500 mL Sakaguchi flask, and autoclaved at 121° C. for 20 minutes. To this cooled liquid medium, the transformant obtained in (3) was inoculated, and shake-cultured at 30° C. for 3 days to obtain a seed culture liquid. 3.5 L of a medium, in which 0.01% (w/v) of riboflavin (NACALAI TESQUE, INC.), 0.005% (w/v) of chloramphenicol (NACALAI TESQUE, INC.) and an antifoaming agent were added to the same composition of the above-mentioned medium, was introduced into a 5 L jar fermentor, and autoclaved at 121° C. for 20 minutes. To this cooled liquid medium, 50 mL of the seed culture liquid was inoculated, and cultured at 30° C., 400 rpm, 1 v/v/m for 3 days. After the culture, the broth was filtered with a filter cloth, the collected filtrate was centrifuged to collect the supernatant, and furthermore filtrated with a membrane filter (10 μm, Advantech Co., Ltd.) to collect the culture supernatant, and concentrated with an ultrafiltration membrane of 8,000 cutoff molecular weight (Millipore Corp.) to obtain a crude enzyme liquid.

The crude enzyme liquid was adjusted to be a 50% saturated ammonium sulfate solution (pH 6.0), left to stand at 4° C. overnight, and then centrifuged to collect a supernatant.

The supernatant was passed through TOYOPEARL Butyl-650C (TOSOH CORPORATION) column previously equilibrated by a 50 mM potassium phosphate buffer (pH 6.0) containing 50% saturated ammonium sulfate to adsorb the enzyme thereto. The column was washed with the same buffer, and then the enzyme was eluted by a gradient elution method from the buffer to 50 mM potassium phosphate buffer (pH 6.0) to collect an active fraction. The collected active fraction was concentrated by an ultrafiltration membrane, desalinated, equilibrated with 1 mM potassium phosphate buffer (pH 6.0), and passed through a DEAE-cellufine A-500m (CHISSO CORPORATION) column previously equilibrated by the same buffer to adsorb the enzyme thereto. The column was washed with the same buffer, and then the enzyme was eluted by a gradient elution method from the buffer to 200 mM potassium phosphate buffer (pH 6.0) to collect an active fraction. A sample obtained by concentrating the collected active fraction with an ultrafiltration membrane of 8,000 cutoff molecular weight, and then performing water substitution was used as the recombinant KcGLD sample. A specific activity of the purified enzyme was 1,200 U/mg.

### Example 5

### Expression of GLD Derived from D. Kabatiella zeae (KzGLD) by Eukaryotic Cell

### (1) Cloning of KzGLD Gene

The KzGLD gene was amplified by PCR using the cDNA library of K. zeae NBRC9664 prepared according to the method described in Example 2 (1) to (3) as a template.

According to the method described in Example 2 (4), PCR in the first step and the second step, the 5′-RACE method and the 3′-RACE method were performed. Finally, PCR was performed using a primer pair of the following primer-KzF and primer-KzR to obtain a DNA fragment containing K. zeae NBRC9664 strain-derived KzGLD gene of whole chain length of 1,776 bp shown in SEQ ID NO: 7. The amino acid sequences encoded by the gene are shown in SEQ ID NO 8.

It should be noted that, in the amino acid sequences of SEQ ID NO 8, a signal sequence was predicted by Signal P4.1, and 16 amino acids of positions 1-16 in the amino acid sequences of SEQ ID NO 8 could be predicted to be the signal sequence.

primer-KzF:
5′-(TGACCAATTCCGCAGCTCGTCAAA)ATGTTGGGTCAATTGGCCGCT
CTCG-3′
(in parentheses: transcription-enhancing factor)
primer-KzR:
5′-CGCTTCTAGAGCATGCTTACTTGTGGCTAGCCTTGATGAG-3′
(underlined: restriction enzyme site (SphI))

### (2) Preparation of Plasmid Vector Containing KzGLD Gene

Using the DNA fragment obtained in (1) as a template, PCR was performed by means of a primer pair of the primer-KzR and primer-GLD-F to amplify the KzGLD gene. According to the method described in Example 2 (5), the amplified KzGLD gene was bonded to the downstream of the promoter to prepare a plasmid vector on which the gene could be expressed. Furthermore, according to the method described in Example 2 (5), the plasmid was extracted and the sequence of the insert in the plasmid was analyzed, and then the KzGLD gene (SEQ ID NO: 7) could be confirmed.

### (3) Acquisition of Transformant

Using the plasmid extracted in (2), a recombinant mold (Aspergillus oryzae) which produces the KzGLD was produced according to the method described in Example 2 (6). The resulting recombinant strain was refined in Czapek-Dox solid medium.

### (4) Confirmation of Recombinant Mold-Derived KzGLD

The activity of the KzGLD was measured according to the method described in Example 2 (7), and then the GLD activity of the present invention could be confirmed.

### Example 6

### Expression of GLD Derived from E. Cladosporium sp. T799 Strain Eukaryotic Cell

### (1) Cloning of Cs7GLD Gene

The Cs7GLD gene was amplified by PCR using the cDNA library of C. Sp. T799 prepared according to the method described in Example 2 (1) to (3) as a template.

According to the method described in Example 2 (4), PCR in the first step and the second step, the 5′-RACE method and the 3′-RACE method were performed. Finally, PCR was performed using a primer pair of the following primer-Cs7F and primer-Cs7R to obtain a DNA fragment containing C. Sp. T799 strain-derived Cs7GLD gene of whole chain length of 1,761 bp shown in SEQ ID NO: 9. The amino acid sequences encoded by the gene are shown in SEQ ID NO: 10.

It should be noted that, in the amino acid sequences of SEQ ID NO 10, a signal sequence was predicted by Signal P4.1, and 17 amino acids of positions 1-17 in the amino acid sequences of SEQ ID NO: 10 could be predicted to be the signal sequence.

primer-Cs7F:
5′-(TGACCAATTCCGCAGCTCGTCAAA)ATGCTGCCACTGCTCGCGACT
CTGG-3′
(in parentheses: transcription-enhancing factor)
primer-Cs7R:
5′-CGCTTCTAGAGCATGCCTAGTTGCACTGCTTAATGCGCTC-3′
(underlined: restriction enzyme site (SphI))

### (2) Preparation of Plasmid Vector Containing Cs7GLD Gene

Using the DNA fragment obtained in (1) as a template, PCR was performed by means of a primer pair of the primer-Cs7R and primer-GLD-F to amplify the Cs7GLD gene. According to the method described in Example 2 (5), the amplified Cs7GLD gene was bonded to the downstream of the promoter to prepare a plasmid vector on which the gene could be expressed. Furthermore, according to the method described in Example 2 (5), the plasmid was extracted and the sequence of the insert in the plasmid was analyzed, and then the Cs7GLD gene (SEQ ID NO 9) could be confirmed.

### (3) Acquisition of Transformant

Using the plasmid extracted in (2), a recombinant mold (Aspergillus oryzae) which produces the Cs7GLD was produced according to the method described in Example 2 (6). The resulting recombinant strain was refined in Czapek-Dox solid medium.

### (4) Confirmation of Recombinant Mold-Derived Cs7GLD

The activity of the Cs7GLD was measured according to the method described in Example 2 (7), and then the GLD activity of the present invention could be confirmed. According to the method described in Example 2 (7), a sample obtained by concentrating the broth by ultrafiltration membrane was used as a recombinant Cs7GLD sample.

### Example 7

### Expression of GLD Derived from F. Fusicladium carpophilum (FcGLD) by Eukaryotic Cell

### (1) Cloning of FcGLD Gene

The FcGLD gene was amplified by PCR using the cDNA library of F. carpophilum NBRC9645 prepared according to the method described in Example 2 (1) to (3) as a template.

According to the method described in Example 2 (4), PCR in the first step and the second step, the 5′-RACE method and the 3′-RACE method were performed. As a result, it was found that the F. carpophilum NBRC9645 strain-derived FcGLD gene included a base sequence of whole chain length of 1,761 bp shown in SEQ ID NO 11. The amino acid sequences encoded by the gene are shown in SEQ ID NO 12.

It should be noted that, in the amino acid sequences of SEQ ID NO 12, a signal sequence was predicted by Signal P4.1, and 17 amino acids of positions 1-17 in the amino acid sequences of SEQ ID NO 12 could be predicted to be the signal sequence.

### (2) Preparation of Plasmid Vector Containing FcGLD Gene

Using the cDNA library prepared in (1) as a template, PCR was performed by means of a primer pair of the primer-FcF and primer-FcR1 to amplify the FcGLD gene in which the termination codon was replaced by TAA. Then, using the PCR product as a template, PCR was performed by means of a primer pair of the primer FcF and primer-FcR2 to amplify a fragment for plasmid insertion.

According to the method described in Example 2 (5), the amplified fragment was bonded to the downstream of the promoter to prepare a plasmid vector on which the FcGLD gene could be expressed. Furthermore, according to the method described in Example 2 (5), the plasmid was extracted and the sequence of the insert in the plasmid was analyzed, and then the FcGLD gene in which the termination codon was replaced by TAA (SEQ ID NO 11) could be confirmed.

primer-FcF:
5′-(CCGCAGCTCGTCAAA)ATGCTCCCGATCCTCGCGTCT-3′
(in parentheses: transcription-enhancing factor)
primer-FcR1:
5′-GTTCAT(TTA)GTGGCTCTCTTGAATGCG-3′
(in parentheses: replaced termination codon)
primer-FcR2:
5′-GTTACGCTTCTAGAGCATGCGTTCAT(TTA)GTGGCTCTC-3′
(underlined: restriction enzyme site (SphI), in
parentheses: replaced termination codon)

### (3) Acquisition of Transformant

Using the plasmid extracted in (2), a recombinant mold (Aspergillus oryzae) which produces the FcGLD was produced according to the method described in Example 2 (6). The resulting recombinant strain was refined in Czapek-Dox solid medium.

### (4) Confirmation of Recombinant Mold-Derived FcGLD

The activity of the FcGLD was measured according to the method described in Example 2 (7), and then the GLD activity of the present invention could be confirmed.

### Pulification of FcGLD

10 mL of a liquid medium consisting of 2% (w/v) of Pinedex (Matsutani Chemical Industry Co., Ltd.), 1% (w/v) of tripton (Becton, Dickinson and Company), 0.5% (w/v) of potassium dihydrogenphosphate (NACALAI TESQUE, INC.), 0.05% (w/v) of magnesium sulfate heptahydrate (NACALAI TESQUE, INC.) and water was introduced into a large test tube (22 mm×200 mm), and autoclaved at 121° C. for 20 minutes. To this cooled liquid medium, the transformant obtained in (4) was inoculated, and shake-cultured at 30° C. for 3 days to obtain a seed culture liquid. 500 mL of a medium, in which 0.01% (w/v) of riboflavin (NACALAI TESQUE, INC.) was added to the same composition of the above-mentioned medium, was introduced into a 200 mL Sakaguchi flask, and autoclaved at 121° C. for 20 minutes. To this cooled liquid medium, 10 mL of the seed culture liquid was inoculated, and shake-cultured at 30° C., 110 rpm for 3 days. After the culture, the broth was filtered with a filter cloth, the collected filtrate was centrifuged to collect the supernatant, and furthermore filtrated with a membrane filter (10 μm, Advantech Co., Ltd.) to collect the culture supernatant, and concentrated with an ultrafiltration membrane of 8,000 cutoff molecular weight (Millipore Corp.) to obtain a crude enzyme liquid.

The crude enzyme liquid was equilibrated with a 5 mM potassium phosphate buffer (pH6.0), and passed through a DEAE-cellufine A-500m (CHISSO CORPORATION) column previously equilibrated by the same buffer to adsorb the enzyme thereto.

The column was washed with the same buffer, and then the enzyme was eluted by a gradient elution method from the buffer to 200 mM potassium phosphate buffer (pH 6.0) to collect an active fraction. A sample obtained by concentrating the collected active fraction with an ultrafiltration membrane of 8,000 cutoff molecular weight, and then performing water substitution was used as the recombinant FcGLD sample. A specific activity of the purified enzyme was 190 U/mg.

### Example 8

### Expression of GLD Derived from G. Cladosporium sp. T806 (Cs8GLD) by Eukaryotic Cell

### (1) Cloning of Cs8GLD Gene

The Cs8GLD gene was amplified by PCR using the cDNA library of C. sp. T806 prepared according to the method described in Example 2 (1) to (3) as a template.

According to the method described in Example 2 (4), PCR in the first step and the second step, the 5′-RACE method and the 3′-RACE method were performed. As a result, it was found that the C. sp. T806 strain-derived Cs8GLD gene included a base sequence of whole chain length of 1,761 bp shown in SEQ ID NO: 13. The amino acid sequences encoded by the gene are shown in SEQ ID NO 14.

It should be noted that, in the amino acid sequences of SEQ ID NO 14, a signal sequence was predicted by Signal P4.1, and 17 amino acids of positions 1-17 in the amino acid sequences of SEQ ID NO: 14 could be predicted to be the signal sequence.

### (2) Preparation of Plasmid Vector Containing Cs8GLD Gene

Using the cDNA library prepared in (1) as a template, PCR was performed by means of a primer pair of the primer-Cs8F and primer-Cs8R1 to amplify the Cs8GLD gene in which the termination codon was replaced by TAA. Then, using the PCR product as a template, PCR was performed by means of a primer pair of the primer-Cs8F and primer-Cs8R2 to amplify a fragment for plasmid insertion. According to the method described in Example 2 (5), the amplified fragment was bonded to the downstream of the promoter to prepare a plasmid vector on which the Cs8GLD gene could be expressed. Furthermore, according to the method described in Example 2 (5), the plasmid was extracted and the sequence of the insert in the plasmid was analyzed, and then the Cs8GLD gene in which the termination codon was replaced by TAA described in SEQ ID NO 13 could be confirmed.

primer-Cs8F:
5′-(CCGCAGCTCGTCAAA)ATGCTCCCAGTGCTCGCGTCT-3′
(in parentheses: transcription-enhancing factor)
primer-Cs8R1:
5′-GTTCAT(TTA)GTGGCTCTGCTGAATACG-3′
(in parentheses: replaced termination codon)
primer-Cs8R2:
5′-((GTTACGCTTCTAGA))GCATGCGTTCAT(TTA)GTGGCTCTG-3′
(underlined: restriction enzyme site (SphI), in
parentheses: replaced termination codon)

### (3) Acquisition of Transformant

Using the plasmid extracted in (2), a recombinant mold (Aspergillus oryzae) which produces the Cs8GLD was produced according to the method described in Example 2 (6). The resulting recombinant strain was refined in Czapek-Dox solid medium.

### (4) Confirmation of Recombinant Mold-Derived Cs8GLD

The activity of the Cs8GLD was measured according to the method described in Example 2 (7), and then the GLD activity of the present invention could be confirmed.

### Example 9

### Expression of GLD Derived from H. Cladosporium cladosporioides (CcGLD) by Eukaryotic Cell

### (1) Cloning of CcGLD Gene

The CcGLD gene was amplified by PCR using the cDNA library of C. cladosporioides NBRC4459 prepared according to the method described in Example 2 (1) to (3) as a template.

According to the method described in Example 2 (4), PCR in the first step and the second step, the 5′-RACE method and the 3′-RACE method were performed. As a result, it was found that the C. cladosporioides NBRC4459 strain-derived CcGLD gene included a base sequence of whole chain length of 1,761 bp shown in SEQ ID NO 15. The amino acid sequences encoded by the gene are shown in SEQ ID NO: 16.

It should be noted that, in the amino acid sequences of SEQ ID NO 16, a signal sequence was predicted by Signal P4.1, and 17 amino acids of positions 1-17 in the amino acid sequences of SEQ ID NO: 16 could be predicted to be the signal sequence.

### (2) Preparation of Plasmid Vector 1 Containing CcGLD Gene

Using the cDNA library prepared in (1) as a template, PCR was performed by means of a primer pair of the primer-CcF and primer-CcR1 to amplify a sequence comprising 1761 bp of the CcGLD gene in which the termination codon was replaced by TAA. Then, using the PCR product as a template, PCR was performed by means of a primer pair of the primer-CcF and primer-CcR2 to amplify a fragment for plasmid insertion. According to the method described in Example 2 (5), the amplified fragment was bonded to the downstream of the promoter to prepare a plasmid vector on which the CcGLD gene could be expressed. Furthermore, according to the method described in Example 2 (5), the plasmid was extracted and the sequence of the insert in the plasmid was analyzed, and then the CcGLD gene in which the termination codon was replaced by TAA of SEQ ID NO 15 could be confirmed. Since this gene is a wild type gene except for the termination codon, it would be referred to as a “wild-type CcGLD gene”.

primer-CcF:
5′-(CCGCAGCTCGTCAAA)ATGCTCCCAATTATCGCGTCT-3′
(in parentheses: transcription-enhancing factor)
primer-CcR1:
5′-GTTCAT(TTA)GTGGCTCTGCTGAATGCGCTC-3′
(in parentheses: replaced termination codon)
primer-CcR2:
5′-GTTACGCTTCTAGAGCATGCGTTCAT(TTA)GTGGCTCTG-3′
(underlined: restriction enzyme site (SphI), in
parentheses: replaced termination codon)

### (3) Preparation of Plasmid Vector 2 Containing CcGLD Gene

Using the signal sequence of the Aspergillus oryzae-derived GLD (Ao signal sequence: SEQ ID NO: 64), a plasmid vector for recombinant production of a mature protein CcGLD on the outside of the fungus cell was prepared. Specifically, a plasmid vector into which a gene modified by replacing a predicted signal sequence coding region of the CcGLD gene by the Ao signal sequence coding region in SEQ ID NO 63 was inserted was prepared.

First, PCR was performed using the cDNA library prepared in (1) as a template by means of a primer pair of the primer-A-CcF mentioned below and the primer-CcR1 mentioned above, and the predicted signal sequence coding region of the CcGLD gene was deleted. Next, PCR was gradually performed in order to add the Ao signal sequence coding region in SEQ ID NO 63, and finally PCR was performed using a primer pair of the primer-A-F and the primer-CcR2. As a result, in the sequences in SEQ ID NO: 15, the base sequences of the positions 1-51 were replaced by 66 bases described in SEQ ID NO 63 to amplify the fragment for insertion of the plasmid in which the termination codon was replaced by TAA.

Subsequently, according to the method described in Example 2 (5), the amplified fragment was bonded to the downstream of the promoter to prepare a plasmid vector on which the modified CcGLD gene could be expressed. Furthermore, according to the method described in Example 2 (5), the plasmid was extracted and the sequence of the insert in the plasmid was analyzed. As a result, 1776 bp of the modified gene in SEQ ID NO 24 could be confirmed, in which the base sequence of positions 1-51 in the sequences described in SEQ ID NO 15 was replaced by 66 bases described in SEQ ID NO 63 and the termination codon was replaced by TAA (1776 bp of positions 1-51 described in SEQ ID NO 15 was fused to the downstream of 66 bp described in SEQ ID NO 63, and the last base was adenine). The gene would be referred to as a “modified CcGLD gene”.

primer-A-CcF:
5′-CCGGCTGGACGGGCCCATTCCACTCCCAGATACGAC-3′
(underlined: Ao signal sequence coding region)
primer-A-F:
5′-(CCGCAGCTCGTCAAA)ATGCTCTTCTCACTGGCATTC-3′
(in parentheses: transcription-enhancing factor,
underlined: Ao signal sequence coding region)

### (4) Acquisition of Transformant

Using each plasmid of the (2) into which the wild-type CcGLD gene was inserted or the (3) into which the modified CcGLD gene was inserted, each recombinant mold (Aspergillus oryzae) containing the wild-type CcGLD gene or the modified CcGLD gene was produced according to the method described in Example 2 (6). Each obtained recombinant strain was refined in Czapek-Dox solid medium.

### (5) Confirmation of Recombinant Mold-Derived CcGLD

When the CcGLD activity derived from each recombinant mold was measured according to the method described in Example 2 (7), the GLD activities of the present invention could be confirmed in both the wild-type gene-derived CcGLD and the modified gene-derived CcGLD, both recombinant molds exhibited productivities equivalent to each other, and both CcGLDs exhibited specific activities equivalent to each other.

### Example 10

### Expression of GLD Derived from I. Cladosporium funiclosum (CfGLD) by Eukaryotic Cell

### (1) Cloning of CfGLD Gene

The CfGLD gene was amplified by PCR using the cDNA library of C. funiclosum NBRC6537 prepared according to the method described in Example 2 (1) to (3) as a template.

According to the method described in Example 2 (4), PCR in the first step and the second step, the 5′-RACE method and the 3′-RACE method were performed. As a result, it was found that the C. funiclosum NBRC6537 strain-derived CfGLD gene included a base sequence of whole chain length of 1,761 bp shown in SEQ ID NO: 17. The amino acid sequences encoded by the gene are shown in SEQ ID NO: 18.

It should be noted that, in the amino acid sequences of SEQ ID NO 18, a signal sequence was predicted by Signal P4.1, and 17 amino acids of positions 1-17 in the amino acid sequences of SEQ ID NO 18 could be predicted to be the signal sequence.

### (2) Preparation of Plasmid Vector Containing CfGLD Gene

Using the signal sequence of the Aspergillus oryzae-derived GLD (Ao signal sequence: SEQ ID NO: 64), a plasmid vector for recombinant production of a mature protein CfGLD on the outside of the fungus cell was prepared. Specifically, a plasmid vector into which a gene modified by replacing a predicted signal sequence coding region of the CfGLD gene by the Ao signal sequence coding region in SEQ ID NO 63 was inserted was prepared.

First, PCR was performed using the cDNA library prepared in (1) as a template by means of a primer pair of the primer-A-CfF mentioned below and the primer-CfR1 mentioned above, and the predicted signal sequence coding region of the CfGLD gene was deleted. Next, PCR was gradually performed in order to add the Ao signal sequence coding region in SEQ ID NO 63, and finally PCR was performed using a primer pair of the above-mentioned primer-A-F and the following primer-CfR2. As a result, in the sequences in SEQ ID NO 17, the base sequences of the positions 1-51 were replaced by 66 bases described in SEQ ID NO 63 to amplify the fragment for insertion of the plasmid in which the termination codon was replaced by TAA.

Subsequently, according to the method described in Example 2 (5), the amplified fragment was bonded to the downstream of the promoter to prepare a plasmid vector on which the modified CfGLD gene could be expressed. Furthermore, according to the method described in Example 2 (5), the plasmid was extracted and the sequence of the insert in the plasmid was analyzed. As a result, 1776 bp of the modified gene in SEQ ID NO 26 could be confirmed, in which the base sequence of positions 1-51 in the sequences described in SEQ ID NO: 17 was replaced by 66 bases described in SEQ ID NO: 63 and the termination codon was replaced by TAA (1776 bp of positions 1-51 described in SEQ ID NO 17 was fused to the downstream of 66 bp described in SEQ ID NO 63, and the last base was adenine). The gene would be referred to as a “modified CfGLD gene”.

primer-A-CfF:
5′-CCGGCTGGACGGGCCCATTCCACTCCTAGATATGAC-3′
(underlined: Ao signal sequence coding region)
primer-CfR1:
5′-GTTCAT(TTA)GTGACTGTGCTGAATACG-3′
(in parentheses: replaced termination codon)
primer-CfR2:
5′-GTTACGCTTCTAGAGCATGCGTTCAT(TTA)GTGACTGTG-3′
(underlined: restriction enzyme site (SphI), in
parentheses: replaced termination codon)

### (3) Acquisition of Transformant

Using a plasmid of the (2) into which the modified CfGLD gene was inserted, a recombinant mold (Aspergillus oryzae) containing the modified CcGLD gene was produced according to the method described in Example 2 (6). The obtained recombinant strain was refined in Czapek-Dox solid medium.

### (4) Confirmation of Recombinant Mold-Derived CcGLD

When the CcGLD activity derived from the modified gene was measured according to the method described in Example 2 (7), the GLD activity of the present invention could be confirmed. The CfGLD exhibited specific activity equivalent to that of the wild-type gene-derived CcGLD described in Example 9.

### Example 11

### Expression of GLD Derived from J. Cladosporium oxysporum (CoGLD) by Eukaryotic Cell

### (1) Cloning of CoGLD Gene

The CoGLD gene was amplified by PCR using the cDNA library of C. oxysporum NBRC32511 prepared according to the method described in Example 2 (1) to (3) as a template.

According to the method described in Example 2 (4), PCR in the first step and the second step, the 5′-RACE method and the 3′-RACE method were performed. As a result, it was found that the C. oxysporum NBRC32511 strain-derived CoGLD gene included a base sequence of whole chain length of 1,761 bp shown in SEQ ID NO: 19. The amino acid sequences encoded by the gene are shown in SEQ ID NO 20.

It should be noted that, in the amino acid sequences of SEQ ID NO: 20, a signal sequence was predicted by Signal P4.1, and 17 amino acids of positions 1-17 in the amino acid sequences of SEQ ID NO: 20 could be predicted to be the signal sequence.

### (2) Preparation of Plasmid Vector 1 Containing CoGLD Gene

Using the cDNA library prepared in (1) as a template, PCR was performed by means of a primer pair of the primer-CoF and primer-CoR1 to amplify a sequence comprising 1761 bp of the CoGLD gene in which the termination codon was replaced by TAA. Then, using the PCR product as a template, PCR was performed by means of a primer pair of the primer-CoF and primer-CoR2 to amplify a fragment for plasmid insertion. According to the method described in Example 2 (5), the amplified fragment was bonded to the downstream of the promoter to prepare a plasmid vector on which the CoGLD gene could be expressed. Furthermore, according to the method described in Example 2 (5), the plasmid was extracted and the sequence of the insert in the plasmid was analyzed, and then the CoGLD gene in which the termination codon was replaced by TAA of SEQ ID NO: 19 could be confirmed. Since this gene is a wild type gene except for the termination codon, it would be referred to as a “wild-type CoGLD gene”.

primer-CoF:
5′-(CCGCAGCTCGTCAAA)ATGCTCCCAGTGCTCGCGTCT-3′
(in parentheses: transcription-enhancing factor)
primer-CoR1:
5′-GTTCAT(TTA)GTGGCTCTGCTGAATACGCTC-3′
(in parentheses: replaced termination codon)
primer-CoR2:
5′-GTTACGCTTCTAGAGCATGCGTTCAT(TTA)GTGGCTCTG-3′
(underlined: restriction enzyme site (SphI), in
parentheses: replaced termination codon)

### (3) Preparation of Plasmid Vector 2 Containing CoGLD Gene

Using the signal sequence of the Aspergillus oryzae-derived GLD (Ao signal sequence: SEQ ID NO 64), a plasmid vector for recombinant production of a mature protein CoGLD on the outside of the fungus cell was prepared. Specifically, a plasmid vector into which a gene modified by replacing a predicted signal sequence coding region of the CoGLD gene by the Ao signal sequence coding region in SEQ ID NO 63 was inserted was prepared.

First, PCR was performed using the cDNA library prepared in (1) as a template by means of a primer pair of the primer-A-CoF mentioned below and the primer-CoR1 mentioned above, and the predicted signal sequence coding region of the CoGLD gene was deleted. Next, PCR was gradually performed in order to add the Ao signal sequence coding region in SEQ ID NO 63, and finally PCR was performed using a primer pair of the above-mentioned primer-A-F and the following primer-CoR2. As a result, in the sequences in SEQ ID NO 19, the base sequences of the positions 1-51 were replaced by 66 bases described in SEQ ID NO 63 to amplify the fragment for insertion of the plasmid in which the termination codon was replaced by TAA.

Subsequently, according to the method described in Example 2 (5), the amplified fragment was bonded to the downstream of the promoter to prepare a plasmid vector on which the modified CoGLD gene could be expressed. Furthermore, according to the method described in Example 2 (5), the plasmid was extracted and the sequence of the insert in the plasmid was analyzed. As a result, 1776 bp of the modified gene in SEQ ID NO 28 could be confirmed, in which the base sequence of positions 1-51 in the sequences described in SEQ ID NO 19 was replaced by 66 bases described in SEQ ID NO 63 and the termination codon was replaced by TAA (1776 bp of positions 1-51 described in SEQ ID NO 19 was fused to the downstream of 66 bp described in SEQ ID NO 63, and the last base was adenine). The gene would be referred to as a “modified CoGLD gene”.

primer-A-CoF:
5′-CCGGCTGGACGGGCCCATTCTACTCCCAGATACGAC-3′
(underlined: Ao signal sequence coding region)

### (4) Acquisition of Transformant

Using each plasmid of the (2) into which the wild-type CoGLD gene was inserted or the (3) into which the modified CoGLD gene was inserted, each recombinant mold (Aspergillus oryzae) containing the wild-type CoGLD gene or the modified CoGLD gene was produced according to the method described in Example 2 (6). Each obtained recombinant strain was refined in Czapek-Dox solid medium.

### (5) Confirmation of Recombinant Mold-Derived CoGLD

When the CoGLD activity derived from each recombinant mold was measured according to the method described in Example 2 (7), the GLD activities of the present invention could be confirmed in both the wild-type gene-derived CoGLD and the modified gene-derived CoGLD, both recombinant molds exhibited productivities equivalent to each other, and both CoGLD exhibited specific activities equivalent to each other.

### Example 12

### N-Terminal Sequence Analysis

### (1) ApsGLD

N-terminal sequence analysis for the purified ApsGLD described in Example 1 was performed. As a result, it was revealed that the amino acid at the N terminal of the enzyme being a mature protein was IPNTL. From this evidence, it is considered that the amino acid sequence of positions 1-16 in the amino acid sequences in SEQ ID NO 2 is the signal sequence, and the mature protein ApsGLD has an amino acid sequence consisting of 575 amino acids of positions 17-591 in SEQ ID NO 2. Furthermore, the base sequence encoding the mature protein is considered to be the base sequence consisting of 1725 bases of positions 49-1776 in SEQ ID NO 1 (including no termination codon).

It should be noted that the signal sequence was coincident with the prediction by Signal P4.1.

### (2) KcGLD

N-terminal sequence analysis for the purified KcGLD described in Example 4 was performed. As a result, it was revealed that the amino acid at the N terminal of the enzyme being a mature protein was STPSR. Consequently, the amino acid sequence of the mature protein was proved to be an amino acid sequence consisting of 569 amino acids of positions 24-592 in the amino acid sequences in SEQ ID NO 6. Furthermore, it turned out that the amino acid sequence of positions 1-23 in the amino acid sequences in SEQ ID NO 6 was the signal sequence, and the signal sequence was cleaved in the process for progression to the mature protein. Additionally, the base sequence encoding the mature protein was proved to be the base sequence consisting of 1707 bases of positions 70-1776 in SEQ ID NO 5 (including no termination codon).

It should be noted that the signal sequence was a sequence being 7 amino acids longer than the sequence expected by Signal P4.1, that means, the expected N terminal was an N terminal to which 7 amino acids were added.

### (3) FcGLD

N-terminal sequence analysis for the purified FcGLD described in Example 7 was performed. As a result, it was revealed that the amino acid at the N terminal of the enzyme being a mature protein was APTVL. Consequently, the amino acid sequence of the mature protein was proved to be an amino acid sequence consisting of 575 amino acids of positions 12-586 in the amino acid sequences in SEQ ID NO 12. Furthermore, it turned out that the amino acid sequence of positions 1-11 in the amino acid sequences in SEQ ID NO 12 was the signal sequence, and the signal sequence was cleaved in the process for progression to the mature protein. Additionally, the base sequence encoding the mature protein was proved to be the base sequence consisting of 1725 bases of positions 34-1758 in SEQ ID NO 11 (including no termination codon).

It should be noted that the signal sequence was a sequence being 6 amino acids shorter than the sequence expected by Signal P4.1, that means, the expected N terminal was an N terminal to which 6 amino acids were deleted.

### (4) ApnGLD

From the analysis result of the ApsGLD in (1) or the KcGLD in (2), the amino acid at the N terminal of the mature protein ApnGLD is considered to be APNTL or STPRY. Thus, the amino acid sequence of the mature protein is considered to be an amino acid sequence consisting of 574 amino acids of positions 16-589 or 567 amino acids of positions 23-589 in the amino acid sequences in SEQ ID NO 4. Furthermore, the amino acid sequence of positions 1-15 or positions 1-22 in the amino acid sequences in SEQ ID NO 4 is considered to be the signal sequence. Additionally, the base sequence encoding the mature protein is considered to be the base sequence consisting of 1722 bases of positions 46-1767 in SEQ ID NO: 3 or 1701 bases of positions 67-1767 in SEQ ID NO 3 (including no termination codon).

### (5) KZGLD

From the analysis result of the ApsGLD in (1) or the KcGLD in (2), the amino acid at the N terminal of the mature protein KzGLD is considered to be IPSTL or HIARY. Thus, the amino acid sequence of the mature protein is considered to be an amino acid sequence consisting of 575 amino acids of positions 17-591 or 568 amino acids of positions 24-591 in the amino acid sequences in SEQ ID NO 8. Furthermore, the amino acid sequence of positions 1-16 or positions 1-23 in the amino acid sequences in SEQ ID NO 8 is considered to be the signal sequence. Additionally, the base sequence encoding the mature protein is considered to be the base sequence consisting of 1725 bases of positions 49-1773 in SEQ ID NO: 7 or 1701 bases of positions 70-1773 in SEQ ID NO 7 (including no termination codon).

### (6) Cs7GLD

From the analysis result of the FcGLD in (3), the amino acid at the N terminal of the mature protein Cs7GLD is considered to be VPASL. Thus, the amino acid sequence of the mature protein is considered to be an amino acid sequence consisting of 575 amino acids of positions 12-586 in the amino acid sequences in SEQ ID NO 10. Furthermore, the amino acid sequence of positions 1-11 in the amino acid sequences in SEQ ID NO 10 is considered to be the signal sequence. Additionally, the base sequence encoding the mature protein is considered to be the base sequence consisting of 1725 bases of positions 34-1725 in SEQ ID NO 9 (including no termination codon).

### (7) Cs8GLD

From the analysis result of the FcGLD in (3), the amino acid at the N terminal of the mature protein Cs8GLD is considered to be APTTL. Thus, the amino acid sequence of the mature protein is considered to be an amino acid sequence consisting of 575 amino acids of positions 12-586 in the amino acid sequences in SEQ ID NO 14. Furthermore, the amino acid sequence of positions 1-11 in the amino acid sequences in SEQ ID NO 14 is considered to be the signal sequence. Additionally, the base sequence encoding the mature protein is considered to be the base sequence consisting of 1725 bases of positions 34-1725 in SEQ ID NO 13 (including no termination codon).

### (8) CcGLD

From the analysis result of the FcGLD in (3), the amino acid at the N terminal of the mature protein CcGLD is considered to be APTAL. Thus, the amino acid sequence of the mature protein is considered to be an amino acid sequence consisting of 575 amino acids of positions 12-586 in the amino acid sequences in SEQ ID NO: 16. Furthermore, the amino acid sequence of positions 1-11 in the amino acid sequences in SEQ ID NO 16 is considered to be the signal sequence. Additionally, the base sequence encoding the mature protein is considered to be the base sequence consisting of 1725 bases of positions 34-1725 in SEQ ID NO 15 (including no termination codon).

### (9) CfGLD

From the analysis result of the FcGLD in (3), the amino acid at the N terminal of the mature protein CfGLD is considered to be APTAL. Thus, the amino acid sequence of the mature protein is considered to be an amino acid sequence consisting of 575 amino acids of positions 12-586 in the amino acid sequences in SEQ ID NO 18. Furthermore, the amino acid sequence of positions 1-11 in the amino acid sequences in SEQ ID NO 18 is considered to be the signal sequence. Additionally, the base sequence encoding the mature protein is considered to be the base sequence consisting of 1725 bases of positions 34-1725 in SEQ ID NO 17 (including no termination codon).

### (10) CoGLD

From the analysis result of the FcGLD in (3), the amino acid at the N terminal of the mature protein CoGLD is considered to be APTAL. Thus, the amino acid sequence of the mature protein is considered to be an amino acid sequence consisting of 575 amino acids of positions 12-586 in the amino acid sequences in SEQ ID NO: 20. Furthermore, the amino acid sequence of positions 1-11 in the amino acid sequences in SEQ ID NO: 20 is considered to be the signal sequence. Additionally, the base sequence encoding the mature protein is considered to be the base sequence consisting of 1725 bases of positions 34-1725 in SEQ ID NO: 19 (including no termination codon).

### (11) Modified Gene-Derived GLD

From the analysis result of the FcGLD in (3), the modified gene-derived GLDs obtained in Examples 9, 10 and 11 are considered to be a modified GLD in which 6 amino acids at N terminal are deleted from a mature protein derived from a wild-type gene.

Namely, since the modified CcGLD gene was designed so that the mature protein obtained from the recombinant consists of 569 amino acids of positions 18-586 in the amino acid sequences in SEQ ID NO: 16, the amino acid sequence of the mature protein is as shown in SEQ ID NO: 25, and the amino acid at the N terminal is HSTPR. Since the wild-type CcGLD is considered to be an amino acid sequence consisting of 575 amino acids of positions 12-586, the modified CcGLD gene-derived GLD is considered to be a modified CcGLD lacking in 6 amino acids at the N terminal.

Similarly, since the modified CfGLD gene was designed so that the mature protein obtained from the recombinant consists of 569 amino acids of positions 18-586 in the amino acid sequences in SEQ ID NO: 18, the amino acid sequence of the mature protein is as shown in SEQ ID NO: 27, and the amino acid at the N terminal is HSTPR. Since the wild-type CfGLD is considered to be an amino acid sequence consisting of 575 amino acids of positions 12-586, the modified CfGLD gene-derived GLD is considered to be a modified CfGLD lacking in 6 amino acids at the N terminal.

Similarly, since the modified CoGLD gene was designed so that the mature protein obtained from the recombinant consists of 569 amino acids of positions 18-586 in the amino acid sequences in SEQ ID NO 20, the amino acid sequence of the mature protein is as shown in SEQ ID NO 29, and the amino acid at the N terminal is HSTPR. Since the wild-type CoGLD is considered to be an amino acid sequence consisting of 575 amino acids of positions 12-586, the modified CoGLD gene-derived GLD is considered to be a modified CoGLD lacking in 6 amino acids at the N terminal.

As shown in Example 9 (5), 10 (4) and 11 (5), even the modified GLD showing deletion at the N terminal had the same activity, the equivalent productivity of the recombinant mold and the equivalent specific activity as of the wild-type GLD.

### Example 13

### Study of the Chemoenzymatic Properties of GLD of the Present Invention

Various properties of respective GLDs obtained in Examples 2 to 7 were evaluated.

### (1) Measurement of Absorption Spectrum

Each GLD was measured for the absorption spectra at 200-700 nm before and after addition of D-glucose using a plate reader (SPECTRA MAX PLUS 384, Molecular Devices, LLC.), and as a result, the absorption maximum shown around 360-380 nm and 450-460 nm disappeared by addition of D-glucose in all GLDs, thus all GLDs of the present invention were proved to be flavin-conjugated proteins.

### (2) Measurement of Glucose Oxidase (GOD) Activity

Search of the GOD activity of each GLD revealed that all GLDs exhibited no GOD activity. Consequently, it was clarified that the GLDs of the present invention were hardly affected by the dissolved oxygen in the reaction system when D-glucose was quantified, because the GLDs of the present invention did not use oxygen as an electron acceptor. The GOD activity was measured by the following method. 1.00 mL of 100 mM potassium phosphate buffer (pH 7.0), 0.10 mL of 25 mM 4-aminoantipyrine, 0.10 mL of 420 mM phenol, 0.10 mL of peroxidase (100 unit/mL), 0.65 mL of ultrapure water, 1.00 mL of D-glucose were mixed, kept at 37° C. for 5 minutes, then 0.05 mL of enzyme sample was added, and the reaction was initiated. From the initiation of the reaction, an increment of the absorbance at 500 nm per one minute (ΔA500) associated with progression of the enzyme reaction was measured to calculate the GLD activity according to Formula 2. In this measurement, for, the GLD activity, an enzyme amount for generating 1 μmol of hydrogen peroxide at 37° C., pH 7.0 per one minute was defined as 1U.

$$GOD activity ( U / mL ) = \frac{( Δ A 500 - Δ A500 blank ) \times 3.0 \times df}{10.66 \times 0.5 \times 1.0 \times 0.05} \qquad [ Formula 2 ]$$

In the formula, 3.0 represents a liquid volume (mL) of reaction reagent+enzyme solution, 10.66 represents a molar extinction coefficient (mM⁻¹ cm⁻¹) in the condition of this measurement, 0.5 represents a production amount of the quinone-type pigment relative to the production amount of 1 mol of hydrogen peroxide, 1.0 represents an optical path length (cm) of a cell, 0.05 represents a liquid volume (mL) of enzyme solution, ΔA500blank represents an increment of the absorbance at 500 nm per one minute in the case that the reaction was initiated by adding a solution used for dilution of the enzyme instead of the enzyme solution, and df represents a dilution ratio.

### (3) Molecular Weight

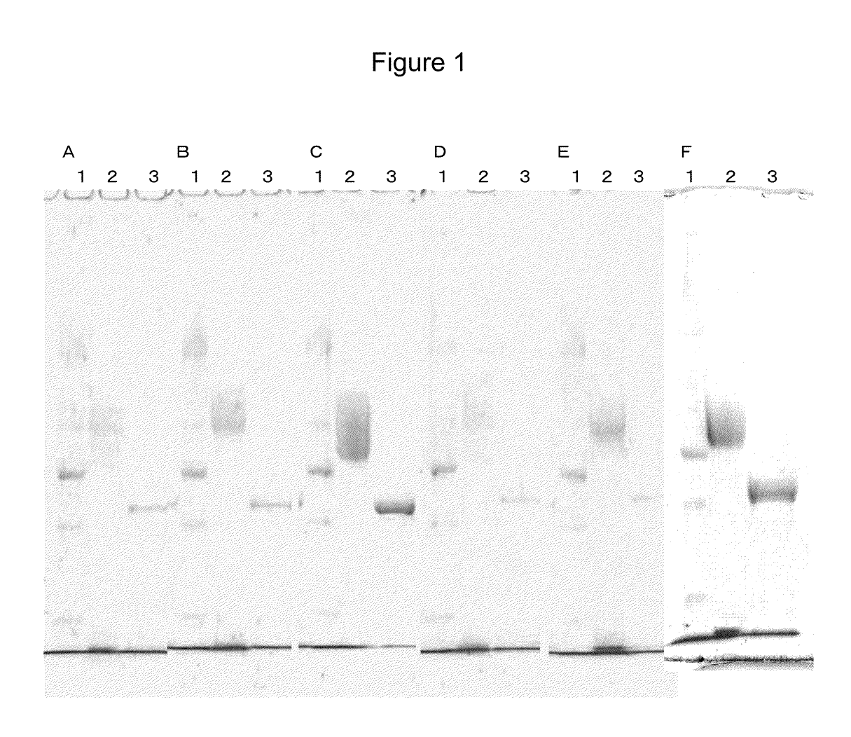

The molecular weights of each GLD before and after cleavage of the sugar chain was calculated by the following method. 5 μL of each GLD solution (respectively prepared to 1.0 mg/mg) and 5 μL of 0.4 M potassium phosphate buffer (pH 6.0) containing 1% of SDS and 2% of β-mercaptoethanol were mixed, heat-treated at 100° C. for 3 minutes. For cleavage treatment of the sugar chain, 10 μL (50 mU) of endoglycosidase H (F. Hoffmann-La Roche Ltd.) was added to the heat-treated sample, and reacted at 37° C. for 18 hours. The sample before and after cleavage of the sugar chain was subjected to SDS-polyacrylamide electrophoresis using 7.5% of e-PAGEL (ATTO Corporation.) to calculate its molecular weight by a molecular weight marker. The results are shown in FIG. 1. The samples for electrophoresis are as below.

### FIG. 1 (A)

Lane 1: Molecular weight marker (DynaMarker Protein Recombinant (10-150 kDa) by BioDynamics Laboratory Inc., 150 kDa, 100 kDa, 80 kDa, 60 kDa and 40 kDa from above.)
Lane 2: Before cleavage of the sugar chain of ApsGLD
Lane 3: After cleavage of the sugar chain of ApsGLD

### FIG. 1 (B)

Lane 1: Molecular weight marker (DynaMarker Protein Recombinant (10-150 kDa) by BioDynamics Laboratory Inc., 150 kDa, 100 kDa, 80 kDa, 60 kDa and 40 kDa from above.)
Lane 2: Before cleavage of the sugar chain of ApnGLD
Lane 3: After cleavage of the sugar chain of ApnGLD

### FIG. 1 (C)

Lane 1: Molecular weight marker (DynaMarker Protein Recombinant (10-150 kDa) by BioDynamics Laboratory Inc., 150 kDa, 100 kDa, 80 kDa, 60 kDa and 40 kDa from above.)

Lane 2: Before cleavage of the sugar chain of KcGLD
Lane 3: After cleavage of the sugar chain of KcGLD

### FIG. 1 (D)

Lane 1: Molecular weight marker (DynaMarker Protein Recombinant (10-150 kDa) by BioDynamics Laboratory Inc., 150 kDa, 100 kDa, 80 kDa, 60 kDa and 40 kDa from above.)
Lane 2: Before cleavage of the sugar chain of KzGLD
Lane 3: After cleavage of the sugar chain of KzGLD

### FIG. 1 (E)

Lane 1: Molecular weight marker (DynaMarker Protein Recombinant (10-150 kDa) by BioDynamics Laboratory Inc., 150 kDa, 100 kDa, 80 kDa, 60 kDa and 40 kDa from above.)
Lane 2: Before cleavage of the sugar chain of Cs7GLD
Lane 3: After cleavage of the sugar chain of Cs7GLD

### FIG. 1 (F)

Lane 1: Molecular weight marker (DynaMarker Protein Recombinant (10-150 kDa) by BioDynamics Laboratory Inc., 150 kDa, 100 kDa, 80 kDa, 60 kDa and 40 kDa from above.)
Lane 2: Before cleavage of the sugar chain of FcGLD
Lane 3: After cleavage of the sugar chain of FcGLD

From FIG. 1, the molecular weight of ApsGLD was 100-115 kDa, the molecular weight of ApnGLD was 95-120 kDa, the molecular weight of KcGLD was 85-115 kDa, the molecular weight of KGLD was 95-115 kDa, the molecular weight of Cs7GLD was 90-105 kDa, and the molecular weight of FcGLD was 85-110 kDa before cleavage of the sugar chain. The respective molecular weight of these GLDs after cleavage of the sugar chain was 60-70 kDa.

### (4) Substrate Specificity

For substrates, D-glucose, maltose, D-galactose, D-fructose, sorbitol, lactose, sucrose, D-xylose, D-mannose and trehalose were respectively used to measure the activity of each GLD corresponding to each substrate according to the method for measuring activity. Relative activity of each substrate when the activity for D-glucose was taken to be 100% was calculated, and the results are shown in Table 1.

TABLE 1

| | Relative activity (%) | | | | | |
|---|---|---|---|---|---|---|
| | (A) | (B) | (C) | (D) | (E) | (F) |
| Substrate | ApsGLD | ApnGLD | KcGLD | KzGLD | CsGLD | FcGLD |
| D-glucose | 100 | 100 | 100 | 100 | 100 | 100 |
| maltose | 5.6 | 4.4 | 2.0 | 2.2 | 3.7 | 2.2 |
| D-xylose | 17 | 17 | 22 | 42 | 36 | 21 |
| D- | 5.2 | 4.1 | 1.0 | 2.4 | 3.3 | 4.5 |
| galactose | | | | | | |
| D-fructose | 0.1> | 0.11 | 0.1> | 0.1> | 0.18 | 0.1> |
| D- | 11 | 8.1 | 4.1 | 3.9 | 8.9 | 7.8 |
| mannose | | | | | | |
| sorbitol | 0.13 | 0.1> | 0.1> | 0.13 | 0.11 | 0.1> |
| lactose | 0.1> | 0.1> | 0.1> | 0.1> | 0.1> | 0.1> |
| trehalose | 22 | 19 | 9.8 | 10 | 22 | 10 |
| sucrose | 0.27 | 0.1> | 0.1> | 0.27 | 0.37 | 0.37 |

When the activity for D-glucose was taken to be 100%, the GLD of the present invention had reactivities of 10% or lower for maltose, D-galactose, D-fructose, sorbitol, lactose and sucrose, and activities of 1% or lower for D-fructose, sorbitol, lactose and sucrose.

### (5) Range of Optimum Temperature

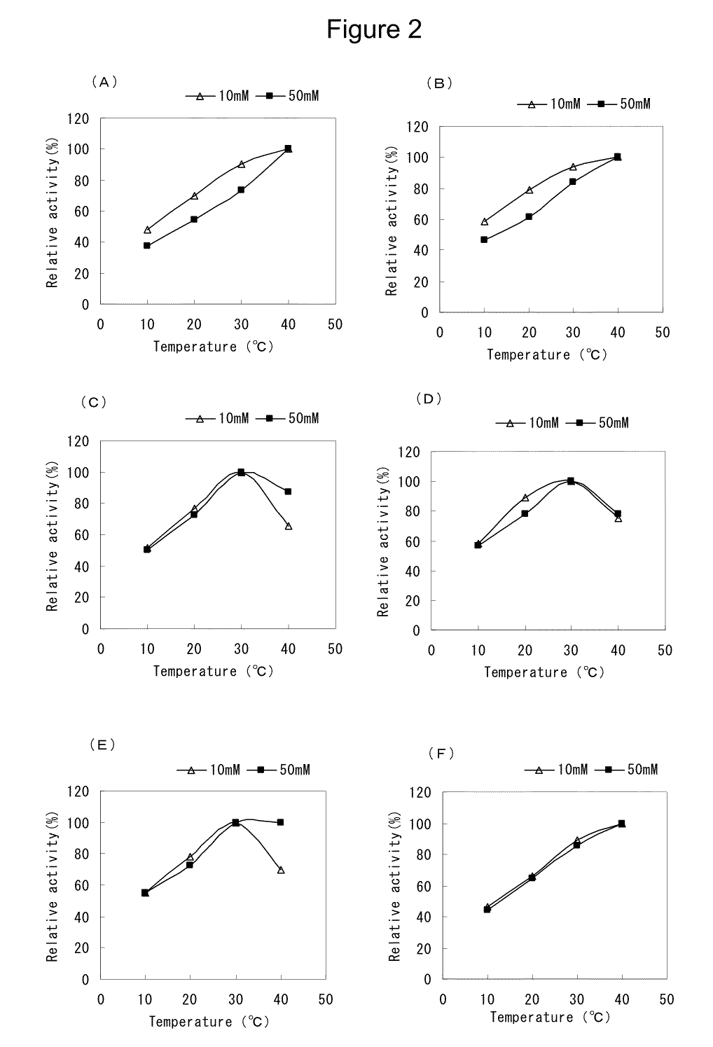

The activity of each GLD was measured at various temperatures according to the method for measuring activity. The final concentrations of the substrate were set to be 10 mM and 50 mM. The results are shown in FIG. 2 (FIG. 2 (A) shows ApsGLD, FIG. 2 (B) shows ApnGLD, FIG. 2 (C) shows KcGLD, FIG. 2 (D) shows KzGLD, FIG. 2 (E) shows Cs7GLD, FIG. 2 (F) shows FcGLD). Specifically, when the final concentrations of the substrate is 10 mM, 1.00 mL of 100 mM potassium phosphate buffer (pH 6.0), 0.03 mL of 1M D-glucose solution, 1.58 mL of ultrapure water, 0.14 mL of 3 mM DCIP and 0.20 mL of 3 mM 1-m-PMS were mixed, and when the final concentrations of the substrate is 50 mM, 1.00 mL of 100 mM potassium phosphate buffer (pH 6.0), 0.15 mL of 1M D-glucose solution, 1.46 mL of ultrapure water, 0.14 mL of 3 mM DCIP and 0.20 mL of 3 mM 1-m-PMS were mixed. In both cases of the final concentrations of the substrate, the mixtures were kept at each temperature instead of 37° C. for 10 minutes, to which 0.05 mL of enzyme sample was added, and the reaction was initiated at each temperature. For 5 minutes from the initiation of the reaction, a decrement of the absorbance at 600 nm per one minute (ΔA600) associated with progression of the enzyme reaction was measured to calculate the GLD activity from a straight part according to the above-described Formula 1. As a result, when an activity value at a temperature where each GLD exhibits the maximum activity was taken to be 100%, at the substrate concentration of 10 mM, the ApsGLD had a relative activity of 80% or higher at 30-40° C., the ApnGLD had of 80% or higher at 30-40° C., the KcGLD had of 80% or higher at 30° C., the KzGLD had of 80% or higher at 20-30° C., the Cs7GLD had of 80% or higher at 30° C., and the FcGLD had of 80% or higher at 30-40° C., and at the substrate concentration of 50 mM, the ApsGLD had a relative activity of 80% or higher at 40° C., the ApnGLD had of 80% or higher at 30-40° C., the KcGLD had of 80% or higher at 30-40° C., the KzGLD had of 80% or higher at 30° C., the Cs7GLD had of 80% or higher at 30-40° C., and the FcGLD had of 80% or higher at 30-40° C., and thus, at the both concentrations, the ApsGLD had the relative activity of 80% or higher at 40° C., the ApnGLD had of 80% or higher at 30-40° C., the KcGLD had of 80% or higher at 30° C., the KzGLD had of 80% or higher at 30° C., the Cs7GLD had of 80% or higher at 30° C., and the FcGLD had of 80% or higher at 30-40° C. From the above, when an activity value at a temperature where the maximum activity was exhibited was taken to be 100%, the optimum temperature for the GLD of the present invention was 30° C. at the substrate concentration of 10 mM, and 30 or 40° C. at the substrate concentration of 50 mM, wherein the relative activity value was 80% or higher.

### (6) Temperature Characteristics

The activity of each GLD was measured at each temperature according to the method for measuring activity. The final concentration of the substrate was 10 mM and 50 mM. The relative activities at each temperature when the activity at 30° C. was taken to be 100% were shown in Table 2. As a result, when an activity value at 30° C. was taken to be 100%, in relation to ranges of the activity values at 10-40° C., at the substrate concentration of 10 mM, the ApsGLD had an activity value ApnGLD had of 62.1-106%, the KcGLD had of 52.0-100%, the KzGLD had of 58.4-100%, the Cs7GLD had of 55.1-100%, and the FcGLD had of 51.6-112%, and at the substrate concentration of 50 mM, the ApsGLD had an activity value of 50.9-136%, the ApnGLD had of 55.9-119%, the KcGLD had of 50.4-100%, the KzGLD had of 56.7-100%, the Cs7GLD had of 55.2-100%, and the FcGLD had of 51.5-117%.

From the above, it turned out that when an activity value at 30° C. was taken to be 100%, the GLD of the present invention had the activity value of 20-150% at 10-40° C., and when an activity value at 30° C. was taken to be 100%, it had the activity value of 20% or higher at 10° C. and 40% or higher at 20° C. Consequently, all of the GLDs of the present invention are enzymes which exhibit small fluctuation in the activity in a wide range of, temperature.

TABLE 2

| | Substrate | Relative activity (%) | | | |
|---|---|---|---|---|---|
| Sample | concentration | 10 | 20 | 30 | 40 |
| (A) | 10 mM | 53.0 | 77.6 | 100 | 111 |
| ApsGLD | 50 mM | 50.9 | 73.7 | 100 | 136 |
| (B) | 10 mM | 62.1 | 83.7 | 100 | 106 |
| ApnGLD | 50 mM | 55.9 | 73.7 | 100 | 119 |
| (C) | 10 mM | 52.0 | 76.9 | 100 | 65.4 |
| KcGLD | 50 mM | 50.4 | 72.9 | 100 | 87.2 |
| (D) | 10 mM | 58.4 | 89.1 | 100 | 75.2 |
| KzGLD | 50 mM | 56.7 | 78.3 | 100 | 77.8 |
| (E) | 10 mM | 55.1 | 78.0 | 100 | 69.8 |
| Cs7GLD | 50 mM | 55.2 | 72.6 | 100 | 99.4 |
| (F) | 10 mM | 51.6 | 74.0 | 100 | 112 |
| FcGLD | 50 mM | 51.5 | 75.9 | 100 | 117 |

### (8) pH Stability

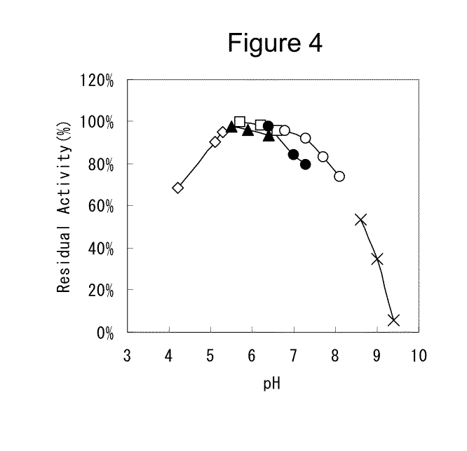

The FcGLD was adjusted to 6 U/mL, and each buffer was added so that the final concentration of a sodium acetate buffer (in the figure, plotted with diamond), a sodium citrate buffer (in the figure, plotted with square), a sodium phosphate buffer (in the figure, plotted with black circle), a potassium phosphate buffer (in the figure, plotted with triangle), a Tris-HCl buffer (in the figure, plotted with white circle) or a glycine-NaOH buffer (in the figure, plotted with cross) was 100 mM respectively, treated at 30° C. for 1 hour, and then the enzyme activity was measured by the method for measuring enzyme activity. A residual ratio of the enzyme activity was calculated, and shown in FIG. 4 as a pH stability.

As a result, when the activity of the enzyme treated with the buffer which exhibited the most stable pH in the FcGLD was taken to be 100%, the remaining activity was 80% or higher at pH 5.0-7.5, and 60% or higher at pH 4.0-8.0. However, even if the buffers were at the same pH, the remaining activities are different depending on the kinds of the buffers, and the potassium phosphate buffer tended to have a lower stability at around pH 7 or alkaline pH compared to other buffers at the same pH.

### (9) Km Value for Glucose

According to the method for measuring activity, the activity of each GLD was measured while changing the concentration of D-glucose as a substrate. From the measured values of the activities at respective glucose concentrations of 5, 15, 25 and 50 mM, Michaelis constants (Km value) were calculated by Hanes-Woolf plot, and as a result, the Km values of the ApsGLD, ApnGLD, KcGLD, KzGLD, Cs7GLD and FcGLD were 8.78 mM, 11.5 mM, 21.6 mM, 37.3 mM, 13.0 mM and 16.8 mM respectively. However, since the Km value easily changes depending on measuring methods and calculated plots, the Km values of the ApsGLD, ApnGLD, KcGLD, KzGLD, Cs7GLD and FcGLD are considered to be about 5-20 mM, about 5-20, about 10-50 mM, about 10-60 mM, about 5-30 mM and about 5-30 mM respectively.

### Example 14

### Measurement of Glucose by the GLD of the Present Invention

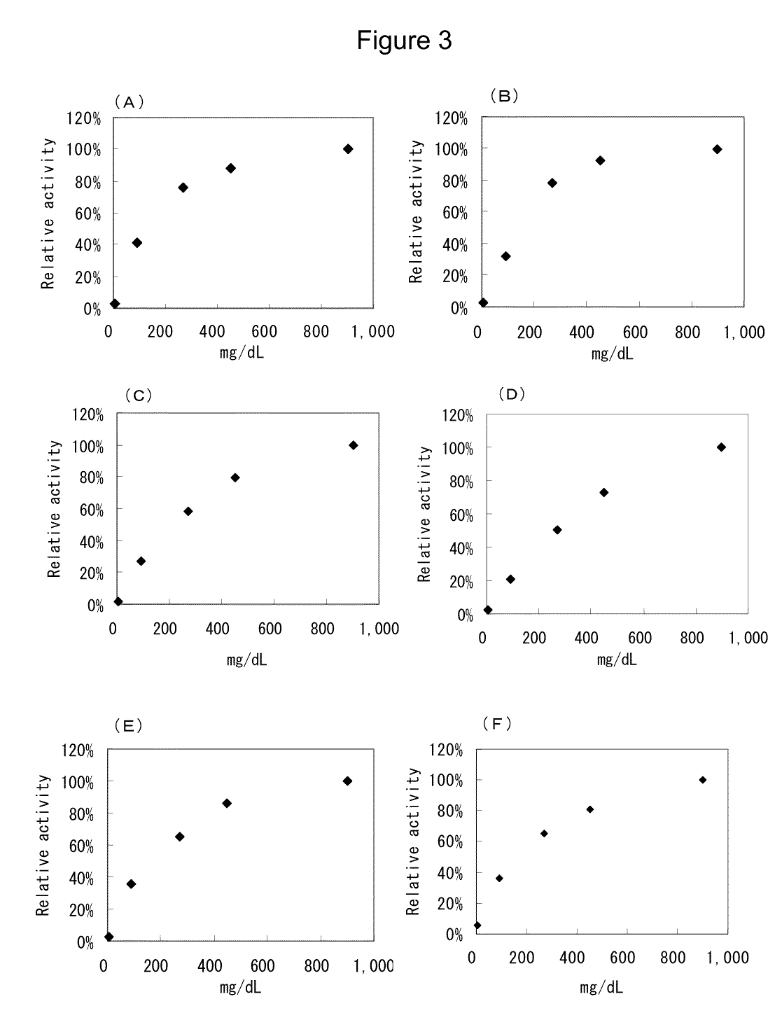

Using the GLD of the present invention, the absorbance change was measured while changing the D-glucose concentration in a range from 0.3 mM (5.5 mg/dL) to 50 mM (900 mg/dL) in the method for measuring activity. The results are shown in FIG. 3 (FIG. 3 (A) shows ApsGLD, FIG. 3 (B) shows ApnGLD, FIG. 3 (C) shows KcGLD, FIG. 3 (D) shows KzGLD, FIG. 3 (E) shows Cs7GLD, FIG. 3 (F) shows FcGLD). As a result, in all GLDs of the present invention, the D-glucose concentration ranging up to 900 mg/dL could be measured. This revealed that D-glucose could be quantified using the GLD of the present invention.

### Example 15

The amino acid sequences of each GLD of the present invention (SEQ ID NO: 2: ApsGLD, 4: ApnGLD, 6: KcGLD, 8: KzGLD, 10: Cs7GLD, 12: FcGLD, 14: Cs8GLD, 16: CcGLD, 18: CfGLD and 20: CoGLD) were compared with each other by homology search of GENETYX, the values of “Similarity %” were organized as similarity % in Table 3, and the values of “identity %” were organized as identity % in Table 4.

Furthermore, the base sequences of each GLD of the present invention (SEQ ID NO: 1: ApsGLD, 3: ApnGLD, 5: KcGLD, 7: KzGLD, 9: Cs7GLD, 11: FcGLD, 13: Cs8GLD, 15: CcGLD, 17: CfGLD and 19: CoGLD) were compared with each other by homology search of GENETYX, and the values of “identity %” were organized as identity % in Table 5.

TABLE 3
Amino acid sequence similarity (%)

| | No. 2 | No. 4 | No. 6 | No. 8 | No. 10 | No. 12 | No. 14 | No. 16 | No. 18 | No. 20 |
|---|---|---|---|---|---|---|---|---|---|---|
| | ApsGLD | ApnGLD | KcGLD | KzGLD | Cs7GLD | FcGLD | Cs8GLD | CcGLD | CfGLD | CoGLD |
| ApsGLD | 100 | 98.1 | 96.2 | 97.4 | 93.6 | 94.6 | 94.6 | 94.7 | 93.8 | 94.3 |
| ApnGLD | 98.1 | 100 | 95.2 | 96.9 | 93.2 | 94.1 | 94.1 | 94.1 | 93.2 | 93.6 |
| KcGLD | 96.2 | 95.2 | 100 | 94.7 | 92.1 | 92.6 | 92.7 | 92.8 | 92.2 | 92.2 |
| KzGLD | 97.4 | 96.9 | 94.7 | 100 | 92.0 | 92.2 | 92.0 | 92.2 | 91.7 | 92.0 |
| Cs7GLD | 93.6 | 93.2 | 92.1 | 92.0 | 100 | 98.1 | 98.1 | 98.2 | 98.1 | 97.7 |
| FcGLD | 94.6 | 94.1 | 92.6 | 92.2 | 98.1 | 100 | 99.8 | 100 | 100 | 99.4 |
| Cs8GLD | 94.6 | 94.1 | 92.7 | 92.0 | 98.1 | 99.8 | 100 | 99.8 | 99.8 | 99.3 |
| CcGLD | 94.7 | 94.1 | 92.8 | 92.2 | 98.2 | 100 | 99.8 | 100 | 100 | 99.4 |
| CfGLD | 93.8 | 93.2 | 92.2 | 91.7 | 98.1 | 100 | 99.8 | 100 | 100 | 99.4 |
| CoGLD | 94.3 | 93.6 | 92.2 | 92.0 | 97.7 | 99.4 | 99.3 | 99.4 | 99.4 | 100 |

TABLE 4
Amino acid sequence identity (%)

| | No. 2 | No. 4 | No. 6 | No. 8 | No. 10 | No. 12 | No. 14 | No. 16 | No. 18 | No. 20 |
|---|---|---|---|---|---|---|---|---|---|---|
| | ApsGLD | ApnGLD | KcGLD | KzGLD | Cs7GLD | FcGLD | Cs8GLD | CcGLD | CfGLD | CoGLD |
| ApsGLD | 100 | 88.4 | 77.1 | 79.5 | 70.4 | 68.5 | 69.0 | 68.3 | 68.7 | 68.9 |
| ApnGLD | 88.4 | 100 | 75.4 | 79.8 | 69.9 | 67.8 | 68.2 | 67.8 | 68.3 | 68.3 |
| KcGLD | 77.1 | 75.4 | 100 | 72.9 | 66.0 | 64.6 | 64.6 | 64.3 | 65.1 | 65.0 |
| KzGLD | 79.5 | 79.8 | 72.9 | 100 | 67.0 | 67.0 | 67.5 | 66.6 | 66.8 | 67.0 |
| Cs7GLD | 70.4 | 69.9 | 66.0 | 67.0 | 100 | 84.4 | 84.6 | 84.8 | 84.6 | 84.1 |
| FcGLD | 68.5 | 67.8 | 64.6 | 67.0 | 84.4 | 100 | 95.5 | 95.7 | 96.0 | 94.7 |
| Cs8GLD | 69.0 | 68.2 | 64.6 | 67.5 | 84.6 | 95.5 | 100 | 96.5 | 94.8 | 95.3 |
| CcGLD | 68.3 | 67.8 | 64.3 | 66.6 | 84.8 | 95.7 | 96.5 | 100 | 95.3 | 95.9 |
| CfGLD | 68.7 | 68.3 | 65.1 | 66.8 | 84.6 | 96.0 | 94.8 | 95.3 | 100 | 95.0 |
| CoGLD | 68.9 | 68.3 | 65.0 | 67.0 | 84.1 | 94.7 | 95.3 | 95.9 | 95.0 | 100 |

TABLE 5
Base sequence identity (%)

| | No. 1 | No. 3 | No. 5 | No. 7 | No. 9 | No. 11 | No. 13 | No. 15 | No. 17 | No. 19 |
|---|---|---|---|---|---|---|---|---|---|---|
| | ApsGLD | ApnGLD | KcGLD | KzGLD | Cs7GLD | FcGLD | Cs8GLD | CcGLD | CfGLD | CoGLD |
| ApsGLD | 100 | 80.9 | 78.4 | 77.0 | 67.9 | 68.6 | 68.3 | 68.0 | 68.6 | 68.5 |
| ApnGLD | 80.9 | 100 | 75.4 | 75.3 | 67.5 | 66.8 | 67.1 | 67.7 | 67.5 | 67.1 |
| KcGLD | 78.4 | 75.4 | 100 | 73.3 | 66.5 | 66.5 | 66.8 | 67.0 | 66.4 | 67.8 |
| KzGLD | 77.0 | 75.3 | 73.3 | 100 | 67.0 | 67.5 | 67.0 | 67.1 | 66.4 | 66.9 |
| Cs7GLD | 67.9 | 67.5 | 66.5 | 67.0 | 100 | 79.7 | 80.5 | 79.9 | 79.8 | 79.7 |
| FcGLD | 68.6 | 66.8 | 66.5 | 67.5 | 79.7 | 100 | 89.0 | 90.1 | 90.9 | 89.6 |
| Cs8GLD | 68.3 | 67.1 | 66.8 | 67.0 | 80.5 | 89.0 | 100 | 92.2 | 89.4 | 90.9 |
| CcGLD | 68.0 | 67.7 | 67.0 | 67.1 | 79.9 | 90.1 | 92.2 | 100 | 89.8 | 90.2 |
| CfGLD | 68.6 | 67.5 | 66.4 | 66.4 | 79.8 | 90.9 | 89.4 | 89.8 | 100 | 89.0 |
| CoGLD | 68.5 | 67.1 | 67.8 | 66.9 | 79.7 | 89.6 | 90.9 | 90.2 | 89.0 | 100 |

From the results in Table 3, it was confirmed that protein which is an amino acid sequence having at least 90% similarity to sequence of respectively SEQ ID NO 2, 4, 6, 8, 10, 12, 14, 16, 18 or 20 and has glucose dehydrogenase activity, and a polynucleotide encoding the protein could be obtained, in the present application.

From the results in Table 4, it was confirmed that protein which is an amino acid sequence having at least 60% identity to sequence of respectively SEQ ID NO: 2, 4, 6, 8, 10, 12, 14, 16, 18 or 20 and has glucose dehydrogenase activity, and a polynucleotide encoding the protein could be obtained, in the present application.

From the results in Table 5, it was confirmed that a polynucleotide encoding protein which is abase sequence having at least 60% identity to sequence of respectively SEQ ID NO 1, 3, 5, 7, 9, 11, 13, 15, 17 or 19 and has glucose dehydrogenase activity could be obtained, in the present application.

1-14. (canceled)

15. A method for manufacturing glucose dehydrogenase, comprising culturing a transformant containing a polynucleotide which encodes a protein having an amino acid sequence of at least 90% similarity with the amino acid sequence shown in SEQ ID NO: 2, and a glucose dehydrogenase activity; and collecting the glucose dehydrogenase from the culture.

16. The method according to claim 15, wherein the polynucleotide which encodes a protein having an amino acid sequence of at least 95% similarity with the amino acid sequence shown in SEQ ID NO: 2, and the glucose dehydrogenase activity.

17. The method according to claim 15, wherein the polynucleotide which encodes a protein having an amino acid sequence of at least 95% similarity with the amino acid sequence shown in SEQ ID NO: 10, and the glucose dehydrogenase activity.

18. The method according to claim 15, wherein the polynucleotide which encodes a protein having an amino acid sequence of at least 90% similarity with the amino acid sequence shown in SEQ ID NO: 2, 4, 6, 8, 10, 12, 14, 16, 18 or 20, and the glucose dehydrogenase activity.

19. A method for producing a biosensor, comprising forming an electrode system on an insulating substrate and providing a measuring reagent containing the glucose dehydrogenase manufactured by the method according to claim 15 and an electron acceptor.

20. The method according to claim 19, wherein the polynucleotide which encodes a protein having an amino acid sequence of at least 95% similarity with the amino acid sequence shown in SEQ ID NO: 2, and the glucose dehydrogenase activity.

21. The method according to claim 19, wherein the polynucleotide which encodes a protein having an amino acid sequence of at least 95% similarity with the amino acid sequence shown in SEQ ID NO: 10, and the glucose dehydrogenase activity.

22. The method according to claim 19, wherein the polynucleotide which encodes a protein having an amino acid sequence of at least 90% similarity with the amino acid sequence shown in SEQ ID NO: 2, 4, 6, 8, 10, 12, 14, 16, 18 or 20, and the glucose dehydrogenase activity.

23. A biosensor for measuring glucose which is provided with a measuring reagent containing the glucose dehydrogenase manufactured by the method according to claim 15 and an electron acceptor; and an electrode system on an insulating substrate.

24. The biosensor according to claim 23, wherein the polynucleotide which encodes a protein having an amino acid sequence of at least 95% similarity with the amino acid sequence shown in SEQ ID NO: 2, and the glucose dehydrogenase activity.

25. The biosensor according to claim 23, wherein the polynucleotide which encodes a protein having an amino acid sequence of at least 95% similarity with the amino acid sequence shown in SEQ ID NO: 10, and the glucose dehydrogenase activity.

26. The biosensor according to claim 23, wherein the polynucleotide which encodes a protein having an amino acid sequence of at least 90% similarity with the amino acid sequence shown in SEQ ID NO: 2, 4, 6, 8, 10, 12, 14, 16, 18 or 20, and the glucose dehydrogenase activity.


SEQUENCE LISTING (64 sequences)

SEQ ID NO: 1 - DNA, 1776 bp, Aureobasidium pullulans S20
  CDS (1)..(1776)
atgttgggtcaactcgctaccctcgcgctcgtttcgactgcttttgctatccccaatactctgcccaaatccacgcctcgttatgattacattattgtgggtggcggcacttccggtttggtcattgccaaccgattgagcgaagatccgaccgtctctgttgctgttattgaggctggcgatcaagtcttcaacaatacgaacgttaccagcgcatccggctatggcaaggcctttggtacagaaattgattgggcatacgagagcgaagctcaggtgtatgccggcaacaagactcagatcttgagagctggtaaggcgctgggaggcaccagcaccatcaatggcatgacttacatgcgtgccgagagcagccagatcgacagctggaagaaggtcggaaacaacatcacctggaactccctgctgccatactacaagaagagtgagtacttcgaataccctactgaggcccaagtctcgatgggcgcatcctacttgcccgaataccacggtaccgaaggcccgttggctgtcagctggcccactgagatggtcggcaacaacttctcaagcatgctcaatgccacattcaaggccatgaagctgccttggaacggagaagcgaacagcggatccatgcgcggatacaacgtcttccccaagacattcgacagatcattagatcttcgtgaggacgccgctcgtgcttattactaccctttcaccacaagacccaaccttgacgtttacctcaactctttcgcacaacgtctgacttggtctaatgacaattcttcggtcgcttttgccaatggtgttgtcttcaccgataagtctggtgccgagcagagcctacttgctaccaaggaagtcgttttgtctgccggatctttgagatctcctcttctgcttgaactctctggtgtcggtaaccctgccgtacttgaaagcctcggcatcgaagtcaaggtcaactctcctttcgtcggcgaaaacctccaggaccagactaccgttgacaccaactacgatgcaactcagaacttcactggtgctggtggctttatcggatacttcaatgctaccgacgtttggggtaatagcaccgcatcatttagcaagactatcaaggcatcgctcgaacagtacgccaacaagactgtacaagcaaccggcggtattaccaacgtcgacactctgctgaggcttttcaacatccagcacgagcttatcttcgaggatgaagttgtcatctctgagatcattgttaacgcaccctcagccagcgctggcttgatcgagtactggggcctgatgcctttctctcgtggaaacattcacatcaagtctgccaacgcctctgctcctgcctccatcaaccccaactacttcctgctcgactatgacatcaagcaacagattggcactgccagaactgccagaaaggttgccactaccgctcctctgagcaacatccttacctccgagacccttcctggcctcgactccgtccctaccaacgcttccgacgccgtctggggtgactggttgaagtcagtctaccgttccaactaccactacatctctactgccgctatgatgtccaaggagctcggtggtgttgtcgacgacaaccacttggtctacggaaccgccaacgttcgcgttgtcgacgcttctgtgctccccttccaggtgtctggccacttgaccagcactttgtatgctcttgccgagagagctgctgatgttatcgaggctagccaccagtag

SEQ ID NO: 2 - protein, 591 aa, Aureobasidium pullulans S20
MLGQLATLALVSTAFAIPNTLPKSTPRYDYIIVGGGTSGLVIANRLSEDPTVSVAVIEAGDQVFNNTNVTSASGYGKAFGTEIDWAYESEAQVYAGNKTQILRAGKALGGTSTINGMTYMRAESSQIDSWKKVGNNITWNSLLPYYKKSEYFEYPTEAQVSMGASYLPEYHGTEGPLAVSWPTEMVGNNFSSMLNATFKAMKLPWNGEANSGSMRGYNVFPKTFDRSLDLREDAARAYYYPFTTRPNLDVYLNSFAQRLTWSNDNSSVAFANGVVFTDKSGAEQSLLATKEVVLSAGSLRSPLLLELSGVGNPAVLESLGIEVKVNSPFVGENLQDQTTVDTNYDATQNFTGAGGFIGYFNATDVWGNSTASFSKTIKASLEQYANKTVQATGGITNVDTLLRLFNIQHELIFEDEVVISEIIVNAPSASAGLIEYWGLMPFSRGNIHIKSANASAPASINPNYFLLDYDIKQQIGTARTARKVATTAPLSNILTSETLPGLDSVPTNASDAVWGDWLKSVYRSNYHYISTAAMMSKELGGVVDDNHLVYGTANVRVVDASVLPFQVSGHLTSTLYALAERAADVIEASHQ

SEQ ID NO: 3 - DNA, 1770 bp, Aureobasidium pullulans NBRC4464
  CDS (1)..(1770)
atgttgggacttgctaccctcgcccttgcgactaccgcattcgctgcccccaacaccctccctaaatcgacgccgcgctatgattatatcatcacagggggtggcctcagtggcttggtcattgccaaccgattgagcgaagacccgaatatctccgtcgctgtcatcgaggccggtgatcaagtcttcaataacacaaatgtgaccagtgcttctggctatggcaaggcctttggcacacagattgattgggcatatgagagtgaagctcaggtctatgctggcaacaagactcaaatcttgagagctggtaaagcgcttggaggcaccagtacaatcaatggaatgacatacatgcgtgctgagagcagtcagatcgacagctggaagaaggttggcaacaacatcacctggtcttctttgctgccatactacaagaagagcgaatattttgagtaccctactgaggctcagatctctatgggcgcatcttacctgcccgagttccacggcactcaaggtcctctctctgtgagctggcccactgagatggtcggcaacaacttctcttccactttgaatgccacattcgaagccctggatctgccttggaacagagatgcaaacagcggatacatgcgtggatacaatgtctttcccagaacgttcgacagagagctggatgttcgtgaggacgctgctcgagcctactactaccctttcaccaccagacccaacctcgatgtctaccttaactcgttcgctcaacgcttgacatggtcgaacgacaactcctcggccgcatttgccaacggtgtcgtatttactgacaagtcaggtaaagagcagaaactcttggctaccaaggaagttatcctatctgccggatcgttgagatcgcctcttcttcttgaactatctggtgttggcaaccccagtgtccttaagaacctcggcatcgaagtcaaggtcaactctccttttgttggagagaacctccaggatcagaccactgtcgacactgactatactgctaacgccaacttcactggcgctggtggtttcatcggatacttcaacgctactgatgtttgggaaaacaacactgcagcattcagcaagaccatcaaggcatctctcgagcaatacgcaaacaggaccgcgcaggctactggcggtattactgaccgtgagactctcctgaaactgttccaaatccagcacgagctcatcttcgaggacgaggttgtcatttcggaggttattgtgaacgcaccctcatctggcagcggcctcctcgagtactggggtcttatgcccttctctcgtggcaacattcacatcaagtctaccaatgcctctgctcctgccgccatcaaccccaacttcttcatgcttgactacgatctcaagcagcagatcggtactgcgagagctgcaagaaaggttgccaccaccgcacctctgagcaacatcctcacttccgagaccactcctggcttcgatgtagttcctctgaacgctactgatgctgtttggggtgactggctgaagtcggtttaccgctccaactaccactacatctccactgctgctatgatgtccaaggagcttggtggagttgttggtgacaaccatctcgtttacggtactgccaatgttcgtgttgtcgatgcctccgtcattcctttccaggtttcgggtcatctggcctcaaccctgtatgctcttgcggagagagctgctgatatcatcaaggccagtcactaa

SEQ ID NO: 4 - protein, 589 aa, Aureobasidium pullulans NBRC4464
MLGLATLALATTAFAAPNTLPKSTPRYDYIITGGGLSGLVIANRLSEDPNISVAVIEAGDQVFNNTNVTSASGYGKAFGTQIDWAYESEAQVYAGNKTQILRAGKALGGTSTINGMTYMRAESSQIDSWKKVGNNITWSSLLPYYKKSEYFEYPTEAQISMGASYLPEFHGTQGPLSVSWPTEMVGNNFSSTLNATFEALDLPWNRDANSGYMRGYNVFPRTFDRELDVREDAARAYYYPFTTRPNLDVYLNSFAQRLTWSNDNSSAAFANGVVFTDKSGKEQKLLATKEVILSAGSLRSPLLLELSGVGNPSVLKNLGIEVKVNSPFVGENLQDQTTVDTDYTANANFTGAGGFIGYFNATDVWENNTAAFSKTIKASLEQYANRTAQATGGITDRETLLKLFQIQHELIFEDEVVISEVIVNAPSSGSGLLEYWGLMPFSRGNIHIKSTNASAPAAINPNFFMLDYDLKQQIGTARAARKVATTAPLSNILTSETTPGFDVVPLNATDAVWGDWLKSVYRSNYHYISTAAMMSKELGGVVGDNHLVYGTANVRVVDASVIPFQVSGHLASTLYALAERAADIIKASH

SEQ ID NO: 5 - DNA, 1779 bp, Kabatiella caulivora
  CDS (1)..(1779)
atgttgggacaagttgctgctctcgcgcttgtttcgactgcctttgctgcgcccaatagtcttcccagatcaactccttctcgttatgactatgttgttgttggtggtggtacctccggcctggtcattgccaaccgattgagtgagaatcccaaagtctctgttgctgtcattgaagctggtggtcaagtcttcaataacaccaacgtcaccagtgtttctggatatggtctggcctttggtacagagattgattgggcatacgagagcgaacctcaggtctatgctggcaacaagccccagactatgagagctggcaaggcgcttggcggtaccagtaccatcaacggtatgacttatttgcgtgcggagagcagccaaatcgacagctggttgaaggtcggaaacaacatcacctgggactcgctacttccttactacaagaaggccgagcaattccaagtcccaacagaggagcaagtcaaggatggtgcatcgtacgatcccgagtttcatggtacccagggaccgttggccgtcggctggccaaatgaaatggttggcggtgactggccatcgcttttgaacaccaccttcaaggctctggatctaccttggaacggagatgccaacgttggatccatgcgtggatatctcatcaaccccaagacatttgacagatccttggatgttcgtgaggatgctgctcgtgcttattactatcctttcgcagcaagatccaaccttcacatctacctccactcgttcgctgaacggctgacatggtcagacggcaacttctcggacgccgttgccaatggtgttgtatacactgatgagtctggcgctgagcagagtatctcggccaccaaggaagtcatcttgtctgccggtgccctaagatctcctcagctcctcgaacactctggtgttggcaaccctactctcctcaacagccttggtattgaagtcaaggttaactctccttttgtcggtgagaacctccaagatcaggctactgtcgacactgcctacgcctccaacgcaagctacgccggttctggtggttacatcggctacttcaatgccaatgatgtctggggcaacggaaccaaagcatacgctgaatccgtcaaggcatccctccaggattgggccaagaagactgcaaacataaccggcggtaccaccaatgcggaagctctgttgaagctgttcgaaatccaacacaagctcatcttcgaggaccaagttgccatctcagaggtcatcgtcatcgcaccctctggcggctccggtcccatcgagtactggggtctgatgcctttctcccgtggaaacattcacatcaagtctgccaaagcctctgacgctgcctccatcaaccccaactactttttcctcgactacgacgtcaagcagcagattgccactgccaaggctgccagaaaggttgctgagactgcacctctgagcagcctcctcacttccgagacccttcctggtcttactactgtccctgaagatgcctccgatgctgtttggggtgattggttgaagtcagcttaccgctcaaacttccactacatctctactgttgccatgatgtccaaggatctcggtggtgttgtcagcgatgagcacttggtttacggaactgccaatgttcgtgttgttgatgcttccgttcttcccttccaggtgtctggtcacttgactagcactttgtatgctcttgctgagagagctgccgatctcatcaaggccaagcacttgtaa

SEQ ID NO: 6 - protein, 592 aa, Kabatiella caulivora
MLGQVAALALVSTAFAAPNSLPRSTPSRYDYVVVGGGTSGLVIANRLSENPKVSVAVIEAGGQVFNNTNVTSVSGYGLAFGTEIDWAYESEPQVYAGNKPQTMRAGKALGGTSTINGMTYLRAESSQIDSWLKVGNNITWDSLLPYYKKAEQFQVPTEEQVKDGASYDPEFHGTQGPLAVGWPNEMVGGDWPSLLNTTFKALDLPWNGDANVGSMRGYLINPKTFDRSLDVREDAARAYYYPFAARSNLHIYLHSFAERLTWSDGNFSDAVANGVVYTDESGAEQSISATKEVILSAGALRSPQLLEHSGVGNPTLLNSLGIEVKVNSPFVGENLQDQATVDTAYASNASYAGSGGYIGYFNANDVWGNGTKAYAESVKASLQDWAKKTANITGGTTNAEALLKLFEIQHKLIFEDQVAISEVIVIAPSGGSGPIEYWGLMPFSRGNIHIKSAKASDAASINPNYFFLDYDVKQQIATAKAARKVAETAPLSSLLTSETLPGLTTVPEDASDAVWGDWLKSAYRSNFHYISTVAMMSKDLGGVVSDEHLVYGTANVRVVDASVLPFQVSGHLTSTLYALAERAADLIKAKHL

SEQ ID NO: 7 - DNA, 1776 bp, Kabatiella zeae
  CDS (1)..(1776)
atgttgggtcaattggccgctctcgctcttgcttctactgctttcgccatccccagcacccttcccaatcacatcgcgcgttatgactacatcatcgttggaggagggtcctcaggtctggtcgttgccaaccgcttgagcgaagacccgaccatctcggtcgctgtcatcgaggccggtgatcaggtgttcaacaggacgaacgtcacaagtgccaccggatacggcaaggactttggcaccgagattgactgggcgtacgagagcgagcctcaaatctatgccggcaacaagtctcagaccttgagagctggcaaggcgctcggaggcacgagcaccatcaatggacagttctacgtccgagccgagaatactcaaatagatgcttgggaaaaggtcggtaacaacctcacctggaactcgctacttccatactacaagaagagtgagtactttgagttccctaccgcggctcaagaagccatgggtgcatcttacgagtccgaataccatggcaccgaaggtcctttggccgttgggtggcctaccgaaatggtcggcggcaacttctccaacattctcaacgccactttcaacgccctgaacctcccttcgaacaaggatctcaacagcggagctatgcgtggatactctgttggcaccaagacttttgatcaatcccttgatgttcgtgaggactctgcccgcgcctactactacccttacgccagcagacccaacttggacatctacctcaactccttcgcccaacgtctgacatggtccaacgagaacgcctcgactcctttcgccgatggtgttgtctttgtcggcaaatctggtcagcagcagaagctgcaggcaaccaaagaagtcatcttgtccgccggttcattgagatccccccttttgctcgagctctctggagtcggcaaccccgccatcctcaaggaactgggtatcgaagtcaaggttgacgcacctttcgtcggcgaaaacctgcaagaccaaaccaccgtcgacacggtctacacctccaaccaaaacatctcgggcgccggaggtttcgtcggatacttcaacgccacggacgtgtggggcaacgccaccgcagcatacagcaagaaggtcaaagcatcgctcgccgactacgccaacaagaccgtcaaagcaacaggcggcaccgccaacgccgaagccctgctgaagctcttcgaaatccagcacgcgcttatcttcgaagacgacgtcgtcatctcagaggtcattgtcaacgcccccgccagcggcagcggcatcgtcgagtactggggtctgatgcccttctcgcgcggaaacatccacatcaagtctgccaacgcatccgcgcccgccgccatcaacccaaactacttccacctcgactttgacatcaagcaacagattgccacggccagaactgcgagaaaagtctctactactgcgcctctgagcaacattttcaccgtcgagaccacgccaggtttcgccgtggtgcccgagaacgcaaccgatgctgtttggggcgactggttgaagtccgtgtaccgctcaaactaccactacattgctacggccgccatgatgtccaaggagctcggcggtgtggttgacgatagccatttggtgtacggcactgccaatgtgagggtggtggatgcttccgttttgccgttccaggtttcgggccatttggcttctacgctttatgcgcttgccgagagagctgctgatctcatcaaggctagccacaagtaa

SEQ ID NO: 8 - protein, 591 aa, Kabatiella zeae
MLGQLAALALASTAFAIPSTLPNHIARYDYIIVGGGSSGLVVANRLSEDPTISVAVIEAGDQVFNRTNVTSATGYGKDFGTEIDWAYESEPQIYAGNKSQTLRAGKALGGTSTINGQFYVRAENTQIDAWEKVGNNLTWNSLLPYYKKSEYFEFPTAAQEAMGASYESEYHGTEGPLAVGWPTEMVGGNFSNILNATFNALNLPSNKDLNSGAMRGYSVGTKTFDQSLDVREDSARAYYYPYASRPNLDIYLNSFAQRLTWSNENASTPFADGVVFVGKSGQQQKLQATKEVILSAGSLRSPLLLELSGVGNPAILKELGIEVKVDAPFVGENLQDQTTVDTVYTSNQNISGAGGFVGYFNATDVWGNATAAYSKKVKASLADYANKTVKATGGTANAEALLKLFEIQHALIFEDDVVISEVIVNAPASGSGIVEYWGLMPFSRGNIHIKSANASAPAAINPNYFHLDFDIKQQIATARTARKVSTTAPLSNIFTVETTPGFAVVPENATDAVWGDWLKSVYRSNYHYIATAAMMSKELGGVVDDSHLVYGTANVRVVDASVLPFQVSGHLASTLYALAERAADLIKASHK

SEQ ID NO: 9 - DNA, 1761 bp, Cladosporium sp. T799
  CDS (1)..(1761)
atgctgccactgctcgcgactctggctactgctgtgccggcctcactacctcactccacgccgcgctacgactacatcatcgtcggtggtggcacgtctggattggtcgtcgcaaacaggctgtctgaggatcctacagtctccgtggccatcattgaggccggtgattctgtctccgacaacgtcaatgtcacgagcgtctctggttatggcaaagcattcggcactcagatcgattgggcgtaccagagcgagcctcaaaagtacgctctgaacaagactcagaccttgatcgctgcgaaagcgctcggtggaaccagcacaatcaacggcatgacctacatgcgtgccgaagacagccagatcgattcatgggcaaagttgggcaacaacatcacatgggactcgctcttgccttactacaagcgcagtgagtacttccaggttcccacagccgcgcaagagtcaacgggagcgtcttatgatgcagcataccacggtcacgatggcccacttgccgttggctggcctaccgagatggtcgaaggaaacttctctggaattctgaatgcaaccttcgcatccctggacctcccatggaacggcgagcccaacaacggacacatgcgtggctacaacatcttccccaagaccgttgaccaggccaacgatgtgagagaggatgctgccagggcttactacttgccaatcagtgatcgtcccaacctggatctttacacgaacgcttttgcacagaggatgacatgggagtccaagtcacacacttcaaagcccttcgcgaacggcgtcgtcttccagtctgccaatggtaccgagaccacgctgctggccactcgcgaagttattctgtccgctggagccttgcgatccccgctccttcttgaattgtctggcgttggaagcaagaccgttcttgagaagcacggcatcaacgtgactgtcaacaatccattcgtcggcgagaacctgcaagaccagacaacaacagacacctcatacaactcgacagaagagtttgccggcgctggaggtttcatcggctactacaacgtcgatgatgtctatggcgatatggctgccaacgtcagcgctactgtcaaccagtccattgcagaatatgctcgcaggaccgccgaagccagcggcgacgtcgtgagcaaagaaactctcgagagattgttccagatccagcacgagatcatcttcaagtacaaggcggtcatctcggaagtcatcgtcaacgcgcctaacagtggtagcgctctcattgaatactggggtcttttgcctttctcgcgcggcaacatccacatccagtctagcaatgcctctgcacccgcctccatcaaccccaactactttatgttggactgggacatgaagcagcagattggcactgcaaagatggctagagcggtcgcgaacactgcgccattcaaagacctccacactgccgaagccctacctggtcttgcggaggtcccggccaacgcttcagatagcgagtgggctgattggttgaagtcgacataccgttccaacttccactacatctcgactgctgctatgatgtcggaggaactcggtggcgttgttgacagtgatcacttggtctacggcacggcgaacgtgcgtgtcgttgatgcttcggtcttgcctttccaggttagcggtcacttgaccagtacgctttatgctttggctgagagagctgccgagcgcattaagcagtgcaactag

SEQ ID NO: 10 - protein, 586 aa, Cladosporium sp. T799
MLPLLATLATAVPASLPHSTPRYDYIIVGGGTSGLVVANRLSEDPTVSVAIIEAGDSVSDNVNVTSVSGYGKAFGTQIDWAYQSEPQKYALNKTQTLIAAKALGGTSTINGMTYMRAEDSQIDSWAKLGNNITWDSLLPYYKRSEYFQVPTAAQESTGASYDAAYHGHDGPLAVGWPTEMVEGNFSGILNATFASLDLPWNGEPNNGHMRGYNIFPKTVDQANDVREDAARAYYLPISDRPNLDLYTNAFAQRMTWESKSHTSKPFANGVVFQSANGTETTLLATREVILSAGALRSPLLLELSGVGSKTVLEKHGINVTVNNPFVGENLQDQTTTDTSYNSTEEFAGAGGFIGYYNVDDVYGDMAANVSATVNQSIAEYARRTAEASGDVVSKETLERLFQIQHEIIFKYKAVISEVIVNAPNSGSALIEYWGLLPFSRGNIHIQSSNASAPASINPNYFMLDWDMKQQIGTAKMARAVANTAPFKDLHTAEALPGLAEVPANASDSEWADWLKSTYRSNFHYISTAAMMSEELGGVVDSDHLVYGTANVRVVDASVLPFQVSGHLTSTLYALAERAAERIKQCN

SEQ ID NO: 11 - DNA, 1761 bp, Fusicladium carpophilum
  CDS (1)..(1761)
atgctcccgatcctcgcgtctctggcagctgccgcgccgactgtattgcctcattccactcctagatacgactacatcatcgtcggcggtggcacttctggtctcgtcgtcgcgaacagactgtccgaggatccctcggtttctgtggctatcattgaagccggcgcttctgcatttgacaacgagaatgtcaccagcgtgtctgcttacggaaaggcattcggcactcagatcgattgggcataccagagtgcgcctcagaagtatgctctcaatgagacgcagactttgagggctggaaaagctcttggtggaacgagcacgttcaatggaatgacctacatgcgtgccgaggacagccagcttgacgcctgggcaaagttgggtaacaatatcacctgggaatcgcttctaccctactacaagcgcagcgagtacttccaggtacctagtgccgcacaagtgtcgatgggagcatcctatgaccctgaataccacggcttcgagggcccccttgccgtcggctggcctaacgagatggtcggtggaaacttctccgccttgctcaacagcactttcgcttcactggacctgccctggaatggcgagcctaacgccggtcacatgcgtggctacaatatcttcccgaagactctcgaccaagcccaggatgtgagggaggattcggcccgtgcttactacttgcccatcagcaaccgtcccaacttggatctttacacgaatgctttcgcgcagagaatgacttgggagacctcgtcgcatactcctaagcctttcgccaacggtgtcatcttcaagtctcctaacggaactgaaactacgctcttcgctactcgtgagatcatcttatctaccggagctttggcttcgccgttgctgcttgagttgtctggagttggaaacaaggctatcctcgagaaaaacggtatcaacgttacagttgacaacgcttttgttggcgagaacctccaagatcaaacaaccaccgacaccacctacaacgcgactacagacttcgccggtgctggtggcttcattggctactacaatgtcgacgatgtctggggcgacatggccgccaacatcagcgcctccgtcaaccagtcgcttgcagagtacgcccgtaagaccgctgaggccagcggcgacatcctcagcgcagagactctcgagaagctgttccgtatccagcacgagttgatcttcaaggacaaggctgtcatctccgaggtcatcgtcaacgctcccaacagcggcagcgccatcctcgagtactggggcctcatgcctttctcccgcggcagcatccacgttcagtctgctaacgcttctgcacctgccgccatcaaccccaactacttcatgctggactgggacatgatgcaacagatcggcaccgcgaagatggcccgcgctgttacgaacactgcgcctttcaagaacctcctcaccggagagactttgcccggcctcgccgaggtctcagccgacgctcctgacagcgagtgggccgcatggttgaagaaaacctaccgttccaacttccactacatctcgactgccgccatgatgtcagaggagcttggcggtgttgtcgacagcgaccacttggtatacggaacggcgaacgttcgtgttgttgatgcttcggtattgccgttccaggttagcggtcacttgactagcactctttatgctttggctgagagggctgcggagcgcattcaagagagccactag

SEQ ID NO: 12 - protein, 586 aa, Fusicladium carpophilum
MLPILASLAAAAPTVLPHSTPRYDYIIVGGGTSGLVVANRLSEDPSVSVAIIEAGASAFDNENVTSVSAYGKAFGTQIDWAYQSAPQKYALNETQTLRAGKALGGTSTFNGMTYMRAEDSQLDAWAKLGNNITWESLLPYYKRSEYFQVPSAAQVSMGASYDPEYHGFEGPLAVGWPNEMVGGNFSALLNSTFASLDLPWNGEPNAGHMRGYNIFPKTLDQAQDVREDSARAYYLPISNRPNLDLYTNAFAQRMTWETSSHTPKPFANGVIFKSPNGTETTLFATREIILSTGALASPLLLELSGVGNKAILEKNGINVTVDNAFVGENLQDQTTTDTTYNATTDFAGAGGFIGYYNVDDVWGDMAANISASVNQSLAEYARKTAEASGDILSAETLEKLFRIQHELIFKDKAVISEVIVNAPNSGSAILEYWGLMPFSRGSIHVQSANASAPAAINPNYFMLDWDMMQQIGTAKMARAVTNTAPFKNLLTGETLPGLAEVSADAPDSEWAAWLKKTYRSNFHYISTAAMMSEELGGVVDSDHLVYGTANVRVVDASVLPFQVSGHLTSTLYALAERAAERIQESH

SEQ ID NO: 13 - DNA, 1761 bp, Cladosporium sp. T806
  CDS (1)..(1761)
atgctcccagtgctcgcgtctctagcagctgccgcgccgaccactttacctcattccactcccagatacgactacatcatcgtcggaggtggcacttctggtcttgttgtcgctaacagactgtccgaggatcccacggtctctgtggctatcattgaggctggcgcttctgcattcgacaaccagaatgtcactagcgtttccgcgtatggaaaggctttcggaacccagatcgattggacataccagagtgcgcctcaaaagtatgctctaaacgagacacagactttgagggctggaaaggctcttggtggaactagcacgttcaatggaatgacctacctgcgtgccgaagacagccagcttgacgcttgggcgaagctgggcaacaacatcacatgggactcgctcctaccttactacaagcgcagcgagtacttccaggtacccactgccgcacaggtctcgatgggagcatcttacgaccctgagtaccacggctttgagggccctctttctgtcggctggcccaatgagatggtcggtggaaacttctctgccttgctcaacagcacctttgcatctctggatctgccttggaacggcgagcccaacgctggccacatgcacggctacaacatcttccccaaaactctcgaccaggctcaagatgtgagagaggattccgcccgtgcttactacttgcccatcagcgaccgtcccaacttggatctttacacgaacgctttcgcgcagaagatgacctgggaaacctcgtcgcacacttccaggcctttcgccaacggtgttgtgttcaagtctgccaacggaactgagactacgctcttcgctactcgtgagatcattctgtctgctggagctttggcttcgccgttgctcctcgagttgtctggcgttggaaacaaggccatcctcgagaaggttggtattaatgttactgtcgacaatgccttcgtcggcgagaacctccaagatcagacgactacagacactacctacaacgcgaccaccgacttcgccggtgctggtggtttcattggctactacaatgtcgatgacgtctggggcgacatggccgccaacatcagcgcttctgtcaaccagtcgcttgcagagtacgcccgcaagaccgtcgaggccagcggcgatatcctcagcgcagagacccttgagaagctgttccgcatccagcacgagttgatcttcaaggacaaggctgtcatctccgaggtcatcgtcaacgctccttccagcggcagtgccattctcgagtactggggcctgatgcctttctcccgcggcaacatccacgttcaatccgccaacgcttccgcacctgctgccatcaacccgaactacttcatgctggactgggacatgatgcagcaaataggcactgcgaagatgtctcgcgcagtcacgaacgctgcacctttcaagaaccttctcactggagagaccctgcctggtctcgctgaggtctcagccaacgctcctgatagcgagtgggccacatggttgaagaagacttaccgctctaacttccactacatctcgactgctgctatgatgtcggaagagcttggaggtgttgttgacagtgatcacctggtttacggaacggccaacgttcgtgttgttgatgcttcggtgttgccgttccaggtcagtggtcacttgactagtacactttatgctttggctgagagggctgcggagcgtattcagcagagccactag

SEQ ID NO: 14 - protein, 586 aa, Cladosporium sp. T806
MLPVLASLAAAAPTTLPHSTPRYDYIIVGGGTSGLVVANRLSEDPTVSVAIIEAGASAFDNQNVTSVSAYGKAFGTQIDWTYQSAPQKYALNETQTLRAGKALGGTSTFNGMTYLRAEDSQLDAWAKLGNNITWDSLLPYYKRSEYFQVPTAAQVSMGASYDPEYHGFEGPLSVGWPNEMVGGNFSALLNSTFASLDLPWNGEPNAGHMHGYNIFPKTLDQAQDVREDSARAYYLPISDRPNLDLYTNAFAQKMTWETSSHTSRPFANGVVFKSANGTETTLFATREIILSAGALASPLLLELSGVGNKAILEKVGINVTVDNAFVGENLQDQTTTDTTYNATTDFAGAGGFIGYYNVDDVWGDMAANISASVNQSLAEYARKTVEASGDILSAETLEKLFRIQHELIFKDKAVISEVIVNAPSSGSAILEYWGLMPFSRGNIHVQSANASAPAAINPNYFMLDWDMMQQIGTAKMSRAVTNAAPFKNLLTGETLPGLAEVSANAPDSEWATWLKKTYRSNFHYISTAAMMSEELGGVVDSDHLVYGTANVRVVDASVLPFQVSGHLTSTLYALAERAAERIQQSH

SEQ ID NO: 15 - DNA, 1761 bp, Cladosporium cladosporioides
  CDS (1)..(1761)
atgctcccaattatcgcgtctctagcggctgccgcccctactgctttgcctcattccactcccagatacgactacatcatcgtcggaggtggcacttctggtcttgtcgtcgctaacagactgtccgaggaccccacggtgtctgtggctatcattgaggctggcgcttctgcattcgacaatgagaatgtgactagcgttgctgcgtacggaaaggctttcggcacccagatcgattgggcgtaccagagtgcgcctcaaaagtatgctctcaacgagacacagactttgagggctggaaaggctcttggtggaactagcacgttcaatggaatgacctacatgcgtgccgaagacagtcagcttgatgcttgggcgaagttgggcaataacatcacctgggactcgcttctcccttactacaagcgcagcgagtacttccaggtacctaccgctgcacaggtctcgatgggcgcctcttatgatcccgagtaccacggcttcgagggtcctctttccgtcggctggcccaacgagatggtcagtggaaacttctccgctttgctcaacagcaccttcgcttctctggatctgccttggaacggcgagcccaacgctggccacatgcgcggctacaacatcttccccaagactcttgaccaggcccaggatgttagggaggattcggctcgtgcttactacttgcccatcagcaaccgtcccaacttggatctttacacgaacgctttcgcgcagaagatgacctgggaaacctcgtcgcacacttccaagcctttcgccaacggtgttgtgttcaagtctgccaacggaactgagactacgctcttcgctactcgtgagatcattctgtctgctggggctttggcttcaccattgctccttgagttgtctggcgtgggaaacaaggctatcctcgagaagaacggcatcaacgtcacagttgacaacgctttcgttggagagaacctccaagatcagacaactaccgacaccacttacaacgcgactaccgacttcgccggtgctggtggtttcatcggctactacaacgtcgatgatgtctggggagacatggccgccaacatcagcgcttccgtcaaccagtcgcttgcagagtacgcccgcaagaccgccaaggccagcggcgatatcctcagcgtagagaccctcgagaagctgttccgcatccagcacgagttgatcttcaaggacaaggctgtcatctccgaggtcatcgtcaacgctccctctagcggcagcgccatcctcgagtactggggcctcatgcctttctcccgcggcaacattcatgttcagtccgccaacgcttctgtgcctgccgctattaaccccaactacttcatgctggactgggacatgatgcagcaaatcggcaccgcgaagatgtcccgcgccgtcacgaacgctgcaccgttcaaggacctcctcactggagagaccctgcccggtcttaccgaggtctcagccaacgcttctgacagcgagtgggccgcatggttgaagaagacttaccgctccaacttccactacatctcgactgccgctatgatgtcggaggagcttggtggtgtcgttgacagcgatcacttggtttatggaacggccaatgttcgtgtcgttgatgcttcggtgctgccattccaggtcagcggtcacttgactagcaccctctatgctttggctgagagggctgcggagcgcattcagcagagccactag

SEQ ID NO: 16 - protein, 586 aa, Cladosporium cladosporioides
MLPIIASLAAAAPTALPHSTPRYDYIIVGGGTSGLVVANRLSEDPTVSVAIIEAGASAFDNENVTSVAAYGKAFGTQIDWAYQSAPQKYALNETQTLRAGKALGGTSTFNGMTYMRAEDSQLDAWAKLGNNITWDSLLPYYKRSEYFQVPTAAQVSMGASYDPEYHGFEGPLSVGWPNEMVSGNFSALLNSTFASLDLPWNGEPNAGHMRGYNIFPKTLDQAQDVREDSARAYYLPISNRPNLDLYTNAFAQKMTWETSSHTSKPFANGVVFKSANGTETTLFATREIILSAGALASPLLLELSGVGNKAILEKNGINVTVDNAFVGENLQDQTTTDTTYNATTDFAGAGGFIGYYNVDDVWGDMAANISASVNQSLAEYARKTAKASGDILSVETLEKLFRIQHELIFKDKAVISEVIVNAPSSGSAILEYWGLMPFSRGNIHVQSANASVPAAINPNYFMLDWDMMQQIGTAKMSRAVTNAAPFKDLLTGETLPGLTEVSANASDSEWAAWLKKTYRSNFHYISTAAMMSEELGGVVDSDHLVYGTANVRVVDASVLPFQVSGHLTSTLYALAERAAERIQQSH

SEQ ID NO: 17 - DNA, 1761 bp, Cladosporium funiculosum
  CDS (1)..(1761)
atgctcccgatcctcgcgtctctggcagctgccgcgccgactgcattgcctcattccactcctagatatgactacatcatcatcggcggtggcacttctggtctagtcgtcgcaaacagactttctgaggacccctcggtctccgtggctatcattgaagccggcgcttccgcatttggcaacgaaaatgtcaccagcgtgtctgcatatggaaaggcttttggcactcagatcgattgggcgtaccagagtgcgcctcagaagtatgctcttaatgagacgcagactttgagggctggaaaagctcttggtggaactagcacgttcaatggaatgacctacatgcgtgccgaggacagccagcttgatgcctgggcaaagctgggcaacaacatcacctgggactcgcttctaccctactacaagcgcagcgagtacttccaggtacccactgccgcacaagtttcgatgggcgcatcttacgatcctgaataccacggcttcgagggccctctggccgtcggctggcccaacgagatggtcggtggcaacttttccgctttactcaacagcacctttgcctctctggatctgccctggaacggcgagcctaacgctggtcacatgcgcggctacaacatcttccccaagactatcgaccaagcccaggatgtgagggaggactcggctcgtgcttactacttgcccatcagcaaccgtcccaacttgaatctttacacgaacgctttcgcgcagaagatgacttgggaaacctcgccgcacacttccaagcctttcgctaacggtgtcgtgttcaagtctcctaacggaactgagactaccctcttcgctactcgtgagatcattttgtctgctggagctttggcttcgccgttgctgcttgagttatctggcgttgggaacaaggccatactcgaaaaaaacggcatcaacgttaccgtcgacaacgccttcgttggcgagaatcttcaagatcagacaaccactgacactacctacaacgcgactactgagttcgccggtgccggcggtttcatcggctactacaacgctgatgatgtctggggtgacatggctgccaacgtcagcgcttctgtcaaccagtcgcttgtagagtacgcccgcaagaccgcggaggccagcggcgatatcctcagtgcagagacccttgagaagctgttccgtatccagcacgagatgatcttcaaggacaaggctgtcatctccgaggtcattgtcaacgctcccaacagcggcagcgccatcctcgagtactggggtcttatgcctttctctcgtggcaacattcacgtccagtctaccaacgcttctgcgcctgccgccatcaaccccaactactttatgttggattgggacatgatgcaacaaatcggcaccgcgaagatggcccgcgcagtcacgaacgctgcacctttcaagaacctcctaaccggagagactctgcccggtctcgctgaggtctcagccgacgcttccgacagcgaatgggccgtatggttgaagaagacttaccgctccaacttccattacatctcgactgctgctatgatgtcggaagagcttggcggtgttgtcgacagcgatcacttggtgtacggaaccgcaaacgttcgtgtcgttgatgcttcggtgttgccgttccaggttagtggtcacctgactagtactctttatgctctggctgagagggccgcggagcgtattcagcacagtcactag

SEQ ID NO: 18 - protein, 586 aa, Cladosporium funiculosum
MLPILASLAAAAPTALPHSTPRYDYIIIGGGTSGLVVANRLSEDPSVSVAIIEAGASAFGNENVTSVSAYGKAFGTQIDWAYQSAPQKYALNETQTLRAGKALGGTSTFNGMTYMRAEDSQLDAWAKLGNNITWDSLLPYYKRSEYFQVPTAAQVSMGASYDPEYHGFEGPLAVGWPNEMVGGNFSALLNSTFASLDLPWNGEPNAGHMRGYNIFPKTIDQAQDVREDSARAYYLPISNRPNLNLYTNAFAQKMTWETSPHTSKPFANGVVFKSPNGTETTLFATREIILSAGALASPLLLELSGVGNKAILEKNGINVTVDNAFVGENLQDQTTTDTTYNATTEFAGAGGFIGYYNADDVWGDMAANVSASVNQSLVEYARKTAEASGDILSAETLEKLFRIQHEMIFKDKAVISEVIVNAPNSGSAILEYWGLMPFSRGNIHVQSTNASAPAAINPNYFMLDWDMMQQIGTAKMARAVTNAAPFKNLLTGETLPGLAEVSADASDSEWAVWLKKTYRSNFHYISTAAMMSEELGGVVDSDHLVYGTANVRVVDASVLPFQVSGHLTSTLYALAERAAERIQHSH

SEQ ID NO: 19 - DNA, 1761 bp, Cladosporium oxysporum
  CDS (1)..(1761)
atgctcccagtgctcgcgtctctggcagctgccgcgccaactgctttgcctcattctactcccagatacgactacattatcgttggcggtggcacttctggtctggtcgtcgctaacagactgtctgaggatcccgcggtctctgtggctatcattgaagctggtgcttctgcatttgacaacgagaacgtcaccagcgtttctgcatacggaaaggctttcggcactgggatcgactgggcgtaccagagtgcgcctcagaagtatgctctcaacgaaacgcagactttgagggctggaaaggctcttggtggaactagcacgttcaatggaatgacttacatgcgtgctgaggacagccagcttgacgcttgggcgaagctgggcaacaacatcacatgggattctctcctgccttactacaagcgcagcgagtacttccaggtacccactgcggcacaagtctcgatgggagcatcatacgaccccgagtaccacggtttcgagggtcctctttccgtcggctggcccaacgagatggtcggtggaaacttctccgccttgctcaacagcaccttcgcttctctggatctgccttggaacggcgagcccaacgctggccacatgcgcggctacaacatcttccccaagactctcgaccaggcccaggatgtcagggaggattcggctcgtgcttactacttgcctatcagcaaccgtcccaacttggatctttacacggacgctttcgcgcagaagatgacttgggagactttatcgcatacctctaagcctttcgctaacggcgttgtgttcaagtctcccaatggaactgagactaagctcttcgctactcgtgagatcatcttatctgctggtgcgttggcttcgccgttgctgcttgagttgtctggcgttggaaacaaggctatcctcgagaagaatggtatcaacgtcacagttgacaatgccttcgtcggcgagaaccttcaagaccagacaaccactgacacgacttacaacgcgaccactgacttcgccggggctggaggtttcattggctactacaatgtcgatgatgtctggggcgacatgtccgccaacgtcagcgtattcgtcaaccagtcgcttgcagagtacgcccgcaagaccgctgaggcgagcggcaacatcctcagcgctgagaccctcgaaaaactgttccgcatccagcacgagatgatcttcaaggacaaggctgtcatctccgaggtcattgtcaacgctccctccagcggtagtgccatcctcgagtactggggactcatgcctttctcccgcggcaacatccacgtccagtccgccaacgcttccgcacctgctgccatcaaccccaactatttcatgctggactgggacatgatgcagcagattggcaccgcaaagatgtcccgcgcagttgcgaacgccgcgcctttcaagacccttctcaccggagagacgctgcctgggctcgctaaggtctcagccaacgcttctgatagcgattgggctgcttggttgaagaagacttaccgctccaacttccactacatctcgactgctgctatgatgtcggaggagcttggcggcgttgtagatagtgatcacttggtttacggaacggccaacgttcgtgtcgtggatgcttcggtgttgccgttccaggtcagtggtcacttgacaagcactctttatgctttggctgagagggctgcggagcgtattcagcagagccactag

SEQ ID NO: 20 - protein, 586 aa, Cladosporium oxysporum
MLPVLASLAAAAPTALPHSTPRYDYIIVGGGTSGLVVANRLSEDPAVSVAIIEAGASAFDNENVTSVSAYGKAFGTGIDWAYQSAPQKYALNETQTLRAGKALGGTSTFNGMTYMRAEDSQLDAWAKLGNNITWDSLLPYYKRSEYFQVPTAAQVSMGASYDPEYHGFEGPLSVGWPNEMVGGNFSALLNSTFASLDLPWNGEPNAGHMRGYNIFPKTLDQAQDVREDSARAYYLPISNRPNLDLYTDAFAQKMTWETLSHTSKPFANGVVFKSPNGTETKLFATREIILSAGALASPLLLELSGVGNKAILEKNGINVTVDNAFVGENLQDQTTTDTTYNATTDFAGAGGFIGYYNVDDVWGDMSANVSVFVNQSLAEYARKTAEASGNILSAETLEKLFRIQHEMIFKDKAVISEVIVNAPSSGSAILEYWGLMPFSRGNIHVQSANASAPAAINPNYFMLDWDMMQQIGTAKMSRAVANAAPFKTLLTGETLPGLAKVSANASDSDWAAWLKKTYRSNFHYISTAAMMSEELGGVVDSDHLVYGTANVRVVDASVLPFQVSGHLTSTLYALAERAAERIQQSH

SEQ ID NO: 21 - protein, 575 aa, Aureobasidium pullulans S20
  CHAIN (1)..(575)
IPNTLPKSTPRYDYIIVGGGTSGLVIANRLSEDPTVSVAVIEAGDQVFNNTNVTSASGYGKAFGTEIDWAYESEAQVYAGNKTQILRAGKALGGTSTINGMTYMRAESSQIDSWKKVGNNITWNSLLPYYKKSEYFEYPTEAQVPMGASYLPEYHGTEGPLAVSWPTEMVGNNFSSMLNATFKAMKLPWNGEANSGSMRGYNVFPKTFDRSLDLREDAARAYYYPFTTRPNLDVYLNSFAQRLTWSNDNSSVAFANGVVFTDKSGAEQSLLATKEVVLSAGSLRSPLLLELSGVGNPAVLESLGIEVKVNSPFVGENLQDQTTVDTNYDATQNFTGAGGFIGYFNATDVWGNSTASFSKTIKASLEQYANKTVQATGGITNVDTLLRLFNIQHELIFEDEVVISEIIVNAPSASAGLIEYWGLMPFSRGNIHIKSADASAPASINPNYFLLDYDIKQQIGTARTARKVATTAPLSNILTSETLPGLDSVPTNASDAVWGDWLKSVYRSNYHYISTAAMMSKELGGVVDDNHLVYGTANVRVVDASVLPFQVSGHLTSTLYALAERAADVIEASHQ

SEQ ID NO: 22 - protein, 569 aa, Kabatiella caulivora
  CHAIN (1)..(569)
STPSRYDYVVVGGGTSGLVIANRLSENPKVSVAVIEAGGQVFNNTNVTSVSGYGLAFGTEIDWAYESEPQVYAGNKPQTMRAGKALGGTSTINGMTYLRAESSQIDSWLKVGNNITWDSLLPYYKKAEQFQVPTEEQVKDGASYDPEFHGTQGPLAVGWPNEMVGGDWPSLLNTTFKALDLPWNGDANVGSMRGYLINPKTFDRSLDVREDAARAYYYPFAARSNLHIYLHSFAERLTWSDGNFSDAVANGVVYTDESGAEQSISATKEVILSAGALRSPQLLEHSGVGNPTLLNSLGIEVKVNSPFVGENLQDQATVDTAYASNASYAGSGGYIGYFNANDVWGNGTKAYAESVKASLQDWAKKTANITGGTTNAEALLKLFEIQHKLIFEDQVAISEVIVIAPSGGSGPIEYWGLMPFSRGNIHIKSAKASDAASINPNYFFLDYDVKQQIATAKAARKVAETAPLSSLLTSETLPGLTTVPEDASDAVWGDWLKSAYRSNFHYISTVAMMSKDLGGVVSDEHLVYGTANVRVVDASVLPFQVSGHLTSTLYALAERAADLIKAKHL

SEQ ID NO: 23 - protein, 575 aa, Fusicladium carpophilum
  CHAIN (1)..(575)
APTVLPHSTPRYDYIIVGGGTSGLVVANRLSEDPSVSVAIIEAGASAFDNENVTSVSAYGKAFGTQIDWAYQSAPQKYALNETQTLRAGKALGGTSTFNGMTYMRAEDSQLDAWAKLGNNITWESLLPYYKRSEYFQVPSAAQVSMGASYDPEYHGFEGPLAVGWPNEMVGGNFSALLNSTFASLDLPWNGEPNAGHMRGYNIFPKTLDQAQDVREDSARAYYLPISNRPNLDLYTNAFAQRMTWETSSHTPKPFANGVIFKSPNGTETTLFATREIILSTGALASPLLLELSGVGNKAILEKNGINVTVDNAFVGENLQDQTTTDTTYNATTDFAGAGGFIGYYNVDDVWGDMAANISASVNQSLAEYARKTAEASGDILSAETLEKLFRIQHELIFKDKAVISEVIVNAPNSGSAILEYWGLMPFSRGSIHVQSANASAPAAINPNYFMLDWDMMQQIGTAKMARAVTNTAPFKNLLTGETLPGLAEVSADAPDSEWAAWLKKTYRSNFHYISTAAMMSEELGGVVDSDHLVYGTANVRVVDASVLPFQVSGHLTSTLYALAERAAERIQESH

SEQ ID NO: 24 - DNA, 1776 bp, Artificial Sequence
  modified gene Aosig66bp(1-66)+CcGLD1709bp(52-1760)+A
atgctcttctcactggcattcctgagtgccctgtcgctggccacggcatcaccggctggacgggcccattccactcccagatacgactacatcatcgtcggaggtggcacttctggtcttgtcgtcgctaacagactgtccgaggaccccacggtgtctgtggctatcattgaggctggcgcttctgcattcgacaatgagaatgtgactagcgttgctgcgtacggaaaggctttcggcacccagatcgattgggcgtaccagagtgcgcctcaaaagtatgctctcaacgagacacagactttgagggctggaaaggctcttggtggaactagcacgttcaatggaatgacctacatgcgtgccgaagacagtcagcttgatgcttgggcgaagttgggcaataacatcacctgggactcgcttctcccttactacaagcgcagcgagtacttccaggtacctaccgctgcacaggtctcgatgggcgcctcttatgatcccgagtaccacggcttcgagggtcctctttccgtcggctggcccaacgagatggtcagtggaaacttctccgctttgctcaacagcaccttcgcttctctggatctgccttggaacggcgagcccaacgctggccacatgcgcggctacaacatcttccccaagactcttgaccaggcccaggatgttagggaggattcggctcgtgcttactacttgcccatcagcaaccgtcccaacttggatctttacacgaacgctttcgcgcagaagatgacctgggaaacctcgtcgcacacttccaagcctttcgccaacggtgttgtgttcaagtctgccaacggaactgagactacgctcttcgctactcgtgagatcattctgtctgctggggctttggcttcaccattgctccttgagttgtctggcgtgggaaacaaggctatcctcgagaagaacggcatcaacgtcacagttgacaacgctttcgttggagagaacctccaagatcagacaactaccgacaccacttacaacgcgactaccgacttcgccggtgctggtggtttcatcggctactacaacgtcgatgatgtctggggagacatggccgccaacatcagcgcttccgtcaaccagtcgcttgcagagtacgcccgcaagaccgccaaggccagcggcgatatcctcagcgtagagaccctcgagaagctgttccgcatccagcacgagttgatcttcaaggacaaggctgtcatctccgaggtcatcgtcaacgctccctctagcggcagcgccatcctcgagtactggggcctcatgcctttctcccgcggcaacattcatgttcagtccgccaacgcttctgtgcctgccgctattaaccccaactacttcatgctggactgggacatgatgcagcaaatcggcaccgcgaagatgtcccgcgccgtcacgaacgctgcaccgttcaaggacctcctcactggagagaccctgcccggtcttaccgaggtctcagccaacgcttctgacagcgagtgggccgcatggttgaagaagacttaccgctccaacttccactacatctcgactgccgctatgatgtcggaggagcttggtggtgtcgttgacagcgatcacttggtttatggaacggccaatgttcgtgtcgttgatgcttcggtgctgccattccaggtcagcggtcacttgactagcaccctctatgctttggctgagagggctgcggagcgcattcagcagagccactaa

SEQ ID NO: 25 - protein, 569 aa, Artificial Sequence
  mature protein CcGLD18-586AA
HSTPRYDYIIVGGGTSGLVVANRLSEDPTVSVAIIEAGASAFDNENVTSVAAYGKAFGTQIDWAYQSAPQKYALNETQTLRAGKALGGTSTFNGMTYMRAEDSQLDAWAKLGNNITWDSLLPYYKRSEYFQVPTAAQVSMGASYDPEYHGFEGPLSVGWPNEMVSGNFSALLNSTFASLDLPWNGEPNAGHMRGYNIFPKTLDQAQDVREDSARAYYLPISNRPNLDLYTNAFAQKMTWETSSHTSKPFANGVVFKSANGTETTLFATREIILSAGALASPLLLELSGVGNKAILEKNGINVTVDNAFVGENLQDQTTTDTTYNATTDFAGAGGFIGYYNVDDVWGDMAANISASVNQSLAEYARKTAKASGDILSVETLEKLFRIQHELIFKDKAVISEVIVNAPSSGSAILEYWGLMPFSRGNIHVQSANASVPAAINPNYFMLDWDMMQQIGTAKMSRAVTNAAPFKDLLTGETLPGLTEVSANASDSEWAAWLKKTYRSNFHYISTAAMMSEELGGVVDSDHLVYGTANVRVVDASVLPFQVSGHLTSTLYALAERAAERIQQSH

SEQ ID NO: 26 - DNA, 1776 bp, Artificial Sequence
  modified gene Aosig66bp(1-66)+CfGLD1709bp(52-1760)+A
atgctcttctcactggcattcctgagtgccctgtcgctggccacggcatcaccggctggacgggcccattccactcctagatatgactacatcatcatcggcggtggcacttctggtctagtcgtcgcaaacagactttctgaggacccctcggtctccgtggctatcattgaagccggcgcttccgcatttggcaacgaaaatgtcaccagcgtgtctgcatatggaaaggcttttggcactcagatcgattgggcgtaccagagtgcgcctcagaagtatgctcttaatgagacgcagactttgagggctggaaaagctcttggtggaactagcacgttcaatggaatgacctacatgcgtgccgaggacagccagcttgatgcctgggcaaagctgggcaacaacatcacctgggactcgcttctaccctactacaagcgcagcgagtacttccaggtacccactgccgcacaagtttcgatgggcgcatcttacgatcctgaataccacggcttcgagggccctctggccgtcggctggcccaacgagatggtcggtggcaacttttccgctttactcaacagcacctttgcctctctggatctgccctggaacggcgagcctaacgctggtcacatgcgcggctacaacatcttccccaagactatcgaccaagcccaggatgtgagggaggactcggctcgtgcttactacttgcccatcagcaaccgtcccaacttgaatctttacacgaacgctttcgcgcagaagatgacttgggaaacctcgccgcacacttccaagcctttcgctaacggtgtcgtgttcaagtctcctaacggaactgagactaccctcttcgctactcgtgagatcattttgtctgctggagctttggcttcgccgttgctgcttgagttatctggcgttgggaacaaggccatactcgaaaaaaacggcatcaacgttaccgtcgacaacgccttcgttggcgagaatcttcaagatcagacaaccactgacactacctacaacgcgactactgagttcgccggtgccggcggtttcatcggctactacaacgctgatgatgtctggggtgacatggctgccaacgtcagcgcttctgtcaaccagtcgcttgtagagtacgcccgcaagaccgcggaggccagcggcgatatcctcagtgcagagacccttgagaagctgttccgtatccagcacgagatgatcttcaaggacaaggctgtcatctccgaggtcattgtcaacgctcccaacagcggcagcgccatcctcgagtactggggtcttatgcctttctctcgtggcaacattcacgtccagtctaccaacgcttctgcgcctgccgccatcaaccccaactactttatgttggattgggacatgatgcaacaaatcggcaccgcgaagatggcccgcgcagtcacgaacgctgcacctttcaagaacctcctaaccggagagactctgcccggtctcgctgaggtctcagccgacgcttccgacagcgaatgggccgtatggttgaagaagacttaccgctccaacttccattacatctcgactgctgctatgatgtcggaagagcttggcggtgttgtcgacagcgatcacttggtgtacggaaccgcaaacgttcgtgtcgttgatgcttcggtgttgccgttccaggttagtggtcacctgactagtactctttatgctctggctgagagggccgcggagcgtattcagcacagtcactaa

SEQ ID NO: 27 - protein, 569 aa, Artificial Sequence
  mature protein CfGLD18-586AA
HSTPRYDYIIIGGGTSGLVVANRLSEDPSVSVAIIEAGASAFGNENVTSVSAYGKAFGTQIDWAYQSAPQKYALNETQTLRAGKALGGTSTFNGMTYMRAEDSQLDAWAKLGNNITWDSLLPYYKRSEYFQVPTAAQVSMGASYDPEYHGFEGPLAVGWPNEMVGGNFSALLNSTFASLDLPWNGEPNAGHMRGYNIFPKTIDQAQDVREDSARAYYLPISNRPNLNLYTNAFAQKMTWETSPHTSKPFANGVVFKSPNGTETTLFATREIILSAGALASPLLLELSGVGNKAILEKNGINVTVDNAFVGENLQDQTTTDTTYNATTEFAGAGGFIGYYNADDVWGDMAANVSASVNQSLVEYARKTAEASGDILSAETLEKLFRIQHEMIFKDKAVISEVIVNAPNSGSAILEYWGLMPFSRGNIHVQSTNASAPAAINPNYFMLDWDMMQQIGTAKMARAVTNAAPFKNLLTGETLPGLAEVSADASDSEWAVWLKKTYRSNFHYISTAAMMSEELGGVVDSDHLVYGTANVRVVDASVLPFQVSGHLTSTLYALAERAAERIQHSH

SEQ ID NO: 28 - DNA, 1776 bp, Artificial Sequence
  modified gene Aosig66bp(1-66)+CoGLD1709bp(52-1760)+A
atgctcttctcactggcattcctgagtgccctgtcgctggccacggcatcaccggctggacgggcccattctactcccagatacgactacattatcgttggcggtggcacttctggtctggtcgtcgctaacagactgtctgaggatcccgcggtctctgtggctatcattgaagctggtgcttctgcatttgacaacgagaacgtcaccagcgtttctgcatacggaaaggctttcggcactgggatcgactgggcgtaccagagtgcgcctcagaagtatgctctcaacgaaacgcagactttgagggctggaaaggctcttggtggaactagcacgttcaatggaatgacttacatgcgtgctgaggacagccagcttgacgcttgggcgaagctgggcaacaacatcacatgggattctctcctgccttactacaagcgcagcgagtacttccaggtacccactgcggcacaagtctcgatgggagcatcatacgaccccgagtaccacggtttcgagggtcctctttccgtcggctggcccaacgagatggtcggtggaaacttctccgccttgctcaacagcaccttcgcttctctggatctgccttggaacggcgagcccaacgctggccacatgcgcggctacaacatcttccccaagactctcgaccaggcccaggatgtcagggaggattcggctcgtgcttactacttgcctatcagcaaccgtcccaacttggatctttacacggacgctttcgcgcagaagatgacttgggagactttatcgcatacctctaagcctttcgctaacggcgttgtgttcaagtctcccaatggaactgagactaagctcttcgctactcgtgagatcatcttatctgctggtgcgttggcttcgccgttgctgcttgagttgtctggcgttggaaacaaggctatcctcgagaagaatggtatcaacgtcacagttgacaatgccttcgtcggcgagaaccttcaagaccagacaaccactgacacgacttacaacgcgaccactgacttcgccggggctggaggtttcattggctactacaatgtcgatgatgtctggggcgacatgtccgccaacgtcagcgtattcgtcaaccagtcgcttgcagagtacgcccgcaagaccgctgaggcgagcggcaacatcctcagcgctgagaccctcgaaaaactgttccgcatccagcacgagatgatcttcaaggacaaggctgtcatctccgaggtcattgtcaacgctccctccagcggtagtgccatcctcgagtactggggactcatgcctttctcccgcggcaacatccacgtccagtccgccaacgcttccgcacctgctgccatcaaccccaactatttcatgctggactgggacatgatgcagcagattggcaccgcaaagatgtcccgcgcagttgcgaacgccgcgcctttcaagacccttctcaccggagagacgctgcctgggctcgctaaggtctcagccaacgcttctgatagcgattgggctgcttggttgaagaagacttaccgctccaacttccactacatctcgactgctgctatgatgtcggaggagcttggcggcgttgtagatagtgatcacttggtttacggaacggccaacgttcgtgtcgtggatgcttcggtgttgccgttccaggtcagtggtcacttgacaagcactctttatgctttggctgagagggctgcggagcgtattcagcagagccactaa

SEQ ID NO: 29 - protein, 569 aa, Artificial Sequence
  mature protein CoGLD18-586AA
HSTPRYDYIIVGGGTSGLVVANRLSEDPAVSVAIIEAGASAFDNENVTSVSAYGKAFGTGIDWAYQSAPQKYALNETQTLRAGKALGGTSTFNGMTYMRAEDSQLDAWAKLGNNITWDSLLPYYKRSEYFQVPTAAQVSMGASYDPEYHGFEGPLSVGWPNEMVGGNFSALLNSTFASLDLPWNGEPNAGHMRGYNIFPKTLDQAQDVREDSARAYYLPISNRPNLDLYTDAFAQKMTWETLSHTSKPFANGVVFKSPNGTETKLFATREIILSAGALASPLLLELSGVGNKAILEKNGINVTVDNAFVGENLQDQTTTDTTYNATTDFAGAGGFIGYYNVDDVWGDMSANVSVFVNQSLAEYARKTAEASGNILSAETLEKLFRIQHEMIFKDKAVISEVIVNAPSSGSAILEYWGLMPFSRGNIHVQSANASAPAAINPNYFMLDWDMMQQIGTAKMSRAVANAAPFKTLLTGETLPGLAKVSANASDSDWAAWLKKTYRSNFHYISTAAMMSEELGGVVDSDHLVYGTANVRVVDASVLPFQVSGHLTSTLYALAERAAERIQQSH

SEQ ID NO: 30 - DNA, 24 bp, Artificial Sequence
  primer-F1
cggcactcagatygaytgggcrta

SEQ ID NO: 31 - DNA, 24 bp, Artificial Sequence
  primer-F2
aagttggghaacaacmtcacmtgg

SEQ ID NO: 32 - DNA, 23 bp, Artificial Sequence
  primer-R1
atgcgctcrgcagctctctcvgc

SEQ ID NO: 33 - DNA, 26 bp, Artificial Sequence
  primer-R2
acgccaccgaghtcctysgacatcat

SEQ ID NO: 34 - DNA, 49 bp, Artificial Sequence
  primer-ApsF
tgaccaattccgcagctcgtcaaaatgtatcgtttactctctacatttg

SEQ ID NO: 35 - DNA, 40 bp, Artificial Sequence
  primer-ApsR
cgcttctagagcatgcctactggtggctagcctcgataac

SEQ ID NO: 36 - DNA, 40 bp, Artificial Sequence
  primer-GLD-F
ctccaagttagtcgactgaccaattccgcagctcgtcaaa

SEQ ID NO: 37 - DNA, 49 bp, Artificial Sequence
  primer-ApnF
tgaccaattccgcagctcgtcaaaatgttgggacttgctaccctcgccc

SEQ ID NO: 38 - DNA, 40 bp, Artificial Sequence
  primer-ApnR
cgcttctagagcatgcttagtgactggccttgatgatatc

SEQ ID NO: 39 - DNA, 49 bp, Artificial Sequence
  primer-KcF
tgaccaattccgcagctcgtcaaaatgttgggacaagttgctgctctcg

SEQ ID NO: 40 - DNA, 40 bp, Artificial Sequence
  primer-KcR
cgcttctagagcatgcttacaagtgcttggccttgatgag

SEQ ID NO: 41 - DNA, 49 bp, Artificial Sequence
  primer-KzF
tgaccaattccgcagctcgtcaaaatgttgggtcaattggccgctctcg

SEQ ID NO: 42 - DNA, 40 bp, Artificial Sequence
  primer-KzR
cgcttctagagcatgcttacttgtggctagccttgatgag

SEQ ID NO: 43 - DNA, 49 bp, Artificial Sequence
  primer-Cs7F
tgaccaattccgcagctcgtcaaaatgctgccactgctcgcgactctgg

SEQ ID NO: 44 - DNA, 40 bp, Artificial Sequence
  primer-Cs7R
cgcttctagagcatgcctagttgcactgcttaatgcgctc

SEQ ID NO: 45 - DNA, 36 bp, Artificial Sequence
  primer-FcF
ccgcagctcgtcaaaatgctcccgatcctcgcgtct

SEQ ID NO: 46 - DNA, 27 bp, Artificial Sequence
  primer-FcR1
gttcatttagtggctctcttgaatgcg

SEQ ID NO: 47 - DNA, 38 bp, Artificial Sequence
  primer-FcR2
gttacgcttctagagcatgcgttcatttagtggctctc

SEQ ID NO: 48 - DNA, 36 bp, Artificial Sequence
  primer-Cs8F
ccgcagctcgtcaaaatgctcccagtgctcgcgtct

SEQ ID NO: 49 - DNA, 27 bp, Artificial Sequence
  primer-Cs8R1
gttcatttagtggctctgctgaatacg

SEQ ID NO: 50 - DNA, 38 bp, Artificial Sequence
  primer-Cs8R2
gttacgcttctagagcatgcgttcatttagtggctctg

SEQ ID NO: 51 - DNA, 36 bp, Artificial Sequence
  primer-CcF
ccgcagctcgtcaaaatgctcccaattatcgcgtct

SEQ ID NO: 52 - DNA, 30 bp, Artificial Sequence
  primer-CcR1
gttcatttagtggctctgctgaatgcgctc

SEQ ID NO: 53 - DNA, 38 bp, Artificial Sequence; identical to SEQ ID NO: 50
  primer-CcR2

SEQ ID NO: 54 - DNA, 36 bp, Artificial Sequence
  primer-A-CcF
ccggctggacgggcccattccactcccagatacgac

SEQ ID NO: 55 - DNA, 36 bp, Artificial Sequence
  primer-A-F
ccgcagctcgtcaaaatgctcttctcactggcattc

SEQ ID NO: 56 - DNA, 36 bp, Artificial Sequence
  primer-A-CfF
ccggctggacgggcccattccactcctagatatgac

SEQ ID NO: 57 - DNA, 27 bp, Artificial Sequence
  primer-CfR1
gttcatttagtgactgtgctgaatacg

SEQ ID NO: 58 - DNA, 38 bp, Artificial Sequence
  primer-CfR2
gttacgcttctagagcatgcgttcatttagtgactgtg

SEQ ID NO: 59 - DNA, 36 bp, Artificial Sequence; identical to SEQ ID NO: 48
  primer-CoF

SEQ ID NO: 60 - DNA, 30 bp, Artificial Sequence
  primer-CoR1
gttcatttagtggctctgctgaatacgctc

SEQ ID NO: 61 - DNA, 38 bp, Artificial Sequence; identical to SEQ ID NO: 50
  primer-CoR2

SEQ ID NO: 62 - DNA, 36 bp, Artificial Sequence
  primer-A-CoF
ccggctggacgggcccattctactcccagatacgac

SEQ ID NO: 63 - DNA, 66 bp, Aspergillus oryzae
atgctcttctcactggcattcctgagtgccctgtcgctggccacggcatcaccggctggacgggcc

SEQ ID NO: 64 - protein, 22 aa, Aspergillus oryzae
MLFSLAFLSALSLATASPAGRA